(19) United States
(12) Patent Application Publication
(10) Pub. No.: US 2026/0182925 A1
(43) Pub. Date: Jul. 2, 2026

(54) SYSTEM AND METHOD FOR DETECTION OF AN ANOMALY OF PHYSIOLOGICAL APPARATUS

(71) Applicants: Jiangsu Yuwell POCTech Biotechnology Co., Ltd., Zhenjiang (CN); Jiangsu Yuekai Biotechnology Co., Ltd., Nanjing (CN); Zhejiang POCTech Co., Ltd., Huzhou (CN)

(72) Inventors: Zhongda GUAN, Nanjing (CN); Zhen LIANG, Nanjing (CN); Guanqun ZHANG, Nanjing (CN)

(21) Appl. No.: 19/433,622

(22) Filed: Dec. 26, 2025

(30) Foreign Application Priority Data
Dec. 27, 2024 (CN) 202411954216.1
Dec. 31, 2024 (CN) 202411992249.5

(51) Int. Cl. A61B 5/00

(52) U.S. Cl. CPC A61B 5/7225; A61B 5/7239; A61B 2560/0276

(57) ABSTRACT

An anomaly detection method for a physiological parameter detection apparatus is provided. The method includes receiving a human vital sign signal indicative of a physiological parameter from the apparatus, and determining, using a filtering model, an estimated vital sign signal at a current moment based on the human vital sign signal at a previous moment. A signal-state vector is generated based on at least one of the human vital sign signal and the estimated vital sign signal. At least one working attribute of the physiological parameter detection apparatus is acquired, and a hardware-state vector is generated based on the working attribute and a corresponding preset threshold. An anomaly in an output of the physiological parameter detection apparatus is determined based on a combined evaluation of the signal-state vector and the hardware-state vector.

### CROSS-REFERENCE TO RELATED APPLICATIONS

This application claims priority to Chinese Patent Application No. 202411954216.1, filed with China National Intellectual Property Administration on Dec. 27, 2024 and entitled “ANOMALY TYPE DETECTION METHOD AND DEVICE FOR PHYSIOLOGICAL PARAMETER DETECTION APPARATUS, AND MEDIUM” and Chinese Patent Application No. 202411992249.5, filed with China National Intellectual Property Administration on Dec. 31, 2024 and entitled “FAULT TYPE DETECTION METHOD AND DEVICE FOR PHYSIOLOGICAL PARAMETER DETECTION APPARATUS, AND MEDIUM,” each of which is incorporated herein by reference in its entirety.

### TECHNICAL FIELD

This application relates to the technical field of apparatus anomaly detection, and in particular, to a system, method, and a medium for detection of an anomaly of a physiological apparatus.

### BACKGROUND

Physiological parameter detection systems are widely used to acquire human vital sign signals indicative of physiological parameters such as blood glucose, heart rate, blood pressure, or blood oxygen levels. In practical use, the acquired signals are often affected by noise, motion artifacts, sensor aging, calibration drift, or environmental interference, which can lead to abnormal signal fluctuations, dropouts, or distortions that do not reflect true physiological conditions. Reliance on raw signal values or static thresholds alone makes it difficult to distinguish genuine physiological changes from signal corruption or transient disturbances.

In addition to signal-level issues, abnormalities may also arise from the operating state of the physiological parameter detection apparatus itself, such as variations in working voltage, current, temperature, or other hardware attributes caused by component degradation, circuit faults, or unstable operating conditions. Existing anomaly detection approaches typically evaluate signal behavior or hardware status in isolation, without correlating signal evolution over time with the real-time operating state of the apparatus. This separation limits detection accuracy and can result in false alarms or missed anomalies, particularly in continuous monitoring scenarios, highlighting the need for more robust anomaly detection techniques that jointly consider both signal characteristics and apparatus operating conditions.

### SUMMARY

To resolve the foregoing technical problem, one or more embodiments of this application provide a method of detecting an anomaly of a physiological apparatus, a system and a medium.

The following technical solutions are used in one or c embodiments of this application:

According to one aspect, this application provides an anomaly detection method for a physiological parameter detection apparatus, the method including:
receiving, from the physiological parameter detection apparatus, a human vital sign signal indicative of a physiological parameter;
determining, using a filtering model, an estimated vital sign signal at a current moment based on the human vital sign signal at a previous moment;
generating a signal-state vector based on at least one of the human vital sign signal and the estimated vital sign signal;
acquiring at least one working attribute of the physiological parameter detection apparatus;
generating a hardware-state vector based on the at least one working attribute and a corresponding preset threshold; and
determining whether an anomaly exists in the output of the physiological parameter detection apparatus based on the signal-state vector and the hardware-state vector.

In a feasible embodiment, the method further includes:

determining, through a constraint model, a predicted signal interval at the current moment based on a mean and a standard deviation of the human vital sign signal within a preset time window. Further, generating the signal-state vector includes:

determining whether the estimated vital sign signal falls within the predicted signal interval.

Furthermore, determining whether the anomaly exists includes:
evaluating a state value of the signal-state vector corresponding to the current moment, and determining that an anomaly exists when the state value is equal to the second state value.

In a feasible embodiment, the at least one working attribute includes a real-time working voltage and a real-time working current of the physiological parameter detection apparatus.

In a feasible embodiment, the real-time working voltage includes a working electrode voltage, a reference electrode voltage, and a counter electrode voltage, and
the preset threshold corresponding to the real-time working voltage includes a first preset voltage threshold and a second preset voltage threshold,

Accordingly, the method further includes:
comparing the working electrode voltage with the first preset voltage threshold, and comparing the reference electrode voltage with the second preset voltage threshold, to determine a first voltage determination result;
determining a second voltage determination result based on the working electrode voltage, the reference electrode voltage, and the counter electrode voltage; and
determining the anomaly in the output of the physiological parameter detection apparatus, when there is an abnormal result in the first voltage determination result or the second voltage determination result.

In a feasible embodiment, a first preset threshold corresponding to the real-time working voltage includes a first preset voltage difference threshold and a second preset voltage difference threshold. Accordingly, determining the second voltage determination result includes:
determining a first voltage difference between the working electrode voltage and the reference electrode voltage,
determining a second voltage difference between the working electrode voltage and the counter electrode voltage; and
determining that the second voltage determination result indicates an abnormality when the first voltage difference is greater than the first preset voltage difference threshold or the second voltage difference is greater than the second preset voltage difference threshold.

In a feasible embodiment, the method further includes:
comparing the real-time working current with a second preset threshold to determine a current determination result,
obtaining a first scope value corresponding to detection sensitivity of the physiological parameter detection apparatus, and obtaining a second scope value corresponding to a to-be-detected physiological parameter;
determining a threshold interval of the real-time working current according to the first scope value and the second scope value, and determining the real-time working current based on the threshold interval, to obtain a first current determination result;
performing signal filtering processing on the real-time working current, and determining a second current determination result according to the processed real-time working current and a preset current threshold;
performing low-pass filtering processing on the real-time working current to obtain a baseline of the real-time working current, and determining a third current determination result based on drift data of the baseline and a corresponding preset drift data threshold; and
determining that the current determination result indicates an abnormality if there is an abnormal result in the first current determination result, the second current determination result, or the third current determination result.

In a feasible embodiment, the method further includes:
determining an anomaly type for the anomaly, the wherein the anomaly type includes a soft anomaly and a hard anomaly. Determining the anomaly type includes:
determining that the anomaly type is the hard anomaly if at least one of the first voltage determination result or the second voltage determination result is an abnormality;
determining that the anomaly type is the hard anomaly if the third current determination result is an abnormality;
detecting whether the abnormality is corrected within a preset time step if the first current determination result or the second current determination result is an abnormality;
determining that the anomaly type is the soft anomaly if the abnormality is corrected within the preset time step; and
determining that the anomaly type is the hard anomaly if the abnormality is not corrected within the preset time step.

In a feasible embodiment, determining the anomaly type further includes:
counting a number of times the physiological parameter detection apparatus has a hard anomaly, and outputting, by the physiological parameter detection apparatus, an alarm signal if the counted number of times exceeds a preset number threshold.

In a feasible embodiment, prior to the obtaining the real-time working voltage and a real-time working current of the physiological parameter detection apparatus, the method further includes:
collecting an initial voltage of the physiological parameter detection apparatus according to a preset sampling period;
calculating an average voltage, a standard deviation, and a range value that correspond to the initial voltage, and determining whether the average voltage, the standard deviation, and the range value are all within corresponding preset thresholds; and
if the average voltage, the standard deviation, and the range value are all within the corresponding preset thresholds, obtaining the real-time working voltage and the real-time working current of the physiological parameter detection apparatus.

In a feasible embodiment, the method further includes:
determining that the real-time working current is an invalid signal if the voltage determination result or the current determination result is an abnormality.

In a feasible embodiment, determining the estimated vital sign signal includes:
linearizing a state equation of the filtering model to obtain a linear state equation; and
obtaining the estimated vital sign signal at the current moment based on an optimal estimation result of the human vital sign signal at the previous moment by using the linear state equation.

In a feasible embodiment, determining the predicted signal interval includes:
calculating a signal standard deviation and a signal mean based on the human vital sign signal within the preset time window; and
inputting the signal standard deviation, the signal mean, the human vital sign signal at the previous moment, and the estimated vital sign signal to the constraint model, and determining, by the constraint model, the predicted signal interval at the current moment.

In a feasible embodiment, the predicted signal interval at the current moment includes a maximum critical value and a minimum critical value;
a calculation formula of the maximum critical value is:

$${upLimit\_CgmSignal}_{i} = G ( {Pro\_CgmSignal}_{i - 1} , Ave\_Pro {\_CgmSignal}_{N} , {\overline{x}}_{k + 1 \vert k} ) + γ * Std\_Pro {\_CgmSignal}_{N}$$

a calculation formula of the minimum critical value is:

$${LowLimit\_CgmSignal}_{i} = G ( {Pro\_CgmSignal}_{i - 1} , Ave\_Pro {\_CgmSignal}_{N} , {\overline{x}}_{k + 1 \vert k} ) - β * Std\_Pro {\_CgmSignal}_{N}$$

wherein Pro_CgmSignalᵢ₋₁ represents the human vital sign signal at the previous moment; Ave_Pro_CgmSignal_{N} represents the signal mean; Std_Pro_CgmSignal_{N} represents the signal standard deviation; and γ and β represent preset coefficients.

In a feasible embodiment, the preset signal value includes a first signal value, a second signal value, and a third signal value,
wherein, before the generating the signal-state vector, the method further includes:
preprocessing the human vital sign signal based on the first signal value, the second signal value, and the third signal value, to obtain a pre-processed vital sign update signal; and
correspondingly, the generating the signal-state vector based on a human vital sign signal and a preset signal value includes:
generating the signal-state vector based on the vital sign update signal.

In a feasible embodiment, preprocessing the human vital sign signal includes:
updating the human vital sign signal to the first signal value if the human vital sign signal is greater than the first signal value;
updating the human vital sign signal to a sum value of the human vital sign signal at the previous moment and the second signal value if the human vital sign signal is greater than the human vital sign signal at the previous moment and a difference is greater than the second signal value; and
updating the human vital sign signal to a difference between the human vital sign signal at the previous moment and the third signal value if the human vital sign signal is less than the human vital sign signal at the previous moment and an absolute value of the difference is greater than the third signal value.

In a feasible embodiment, generating the signal-state vector includes:
determining a state value corresponding to the vital sign update signal as a first preset value if the human vital sign signal is greater than or equal to the first signal value or if the vital sign update signal is 0;
determining the state value corresponding to the vital sign update signal as the first preset value if the human vital sign signal is greater than the human vital sign signal at the previous moment and an absolute value of a difference is greater than or equal to the second signal value;
determining the state value corresponding to the vital sign update signal as the first preset value if the human vital sign signal is less than the human vital sign signal at the previous moment and the absolute value of the difference is greater than or equal to the third signal value;
determining the state value corresponding to the vital sign update signal as a second preset value if the human vital sign signal is not less than the human vital sign signal at the previous moment, or the absolute value of the difference is less than the third signal value; and
generating the signal-state vector based on the state value of each vital sign update signal.

In a feasible embodiment, generating the signal-state vector includes:
determining a state value corresponding to the estimated vital sign signal as a second preset value if the estimated vital sign signal falls within the predicted signal interval;
determining the state value corresponding to the estimated vital sign signal as a first preset value if the estimated vital sign signal does not fall within the predicted signal interval; and
generating the signal-state vector based on the state value of each estimated vital sign signal.

In a feasible embodiment, determining the anomaly includes:
if the state value of the signal-state vector corresponding to the estimated vital sign signal is a first preset value or a state value of the hardware-state vector is the first preset value, determining that an anomaly exists in the human vital sign signal at the current moment; and
if the state value of the signal-state vector corresponding to the estimated vital sign signal is a second preset value and the state value of the hardware-state vector is the second preset value, determining that the human vital sign signal at the current moment is a valid code.

In a feasible embodiment, the method further includes:
determining an optimal estimation result corresponding to the human vital sign signal at the current moment, and acquiring a number of consecutive occurrences of determining that the human vital sign signal at the current moment is a valid code;
outputting the human vital sign signal at the current moment as an unrepairable signal if the number of consecutive occurrences is greater than or equal to a third preset value; and
outputting the human vital sign signal at the current moment as a repairable signal if the number of consecutive occurrences is less than the third preset value.

In a feasible embodiment, generating the signal-state vector includes:
determining a state value corresponding to the vital sign update signal as a first preset value if the vital sign update signal is different from the human vital sign signal, or if the human vital sign signal is zero;
determining the state value corresponding to the vital sign update signal as a second preset value if the vital sign update signal is equal to the human vital sign signal; and
generating the signal-state vector based on the state value corresponding to each vital sign update signal.

According to another aspect, this application further provides a system for detecting an anomaly of a physiological apparatus, the system including:
at least one sensor configured to acquire a working attribute of the physiological parameter detection apparatus;
a processor communicatively coupled to the at least one sensor; and
a memory communicatively coupled to the processor, the memory storing processor-executable instructions, that when executed by the processor, cause the processor to perform the method of detecting an anomaly of a physiological apparatus according to any one of the foregoing items.

According to another aspect, this application further provides a non-volatile computer storage medium, storing a computer-executable instruction, where the computer-executable instruction is set to be capable of performing the method of detecting an anomaly of a physiological apparatus according to any one of the foregoing items.

At least one of the above technical solutions employed in the embodiments of this application can achieve the following beneficial effects:

In this application, by continuously monitoring one or more working attributes of the apparatus, including real-time working voltage and real-time working current, early and reliable detection of anomalies in outputs of a physiological parameter detection apparatus can be achieved. Further, by comparing the working attributes with corresponding preset thresholds, changes in the operating state of the apparatus can be captured in a timely manner, allowing abnormal operating conditions to be identified at an early stage. The real-time, automated evaluation reduces reliance on manual inspection or post-processing analysis, lowers the likelihood of human error, and prevents invalid or unreliable signals generated under faulty apparatus conditions from being continuously used. As a result, continuity, stability, and overall reliability of physiological parameter monitoring are improved, particularly in scenarios requiring long-term or continuous operation, while also providing a basis for targeted maintenance or corrective actions. Furthermore, by jointly analyzing signal-level behavior and apparatus-level operating conditions, accuracy and robustness of anomaly detection is improved. By constructing a signal-state vector based on both a human vital sign signal and an estimated vital sign signal, and by determining signal validity using a predicted signal interval derived from a filtering model and a constraint model, the invention comprehensively accounts for both temporal signal trends and statistically expected signal ranges. The said dual-vector approach enables more precise identification of abnormal signals or anomalies that may not be detectable through simple thresholding of raw data. Moreover, the use of real-time estimation and adaptive constraint modeling allows anomaly detection without dependence on large volumes of historical data, thereby improving responsiveness and timeliness while maintaining high detection accuracy under dynamic physiological and operational conditions.

### BRIEF DESCRIPTION OF THE DRAWINGS

To describe technical solutions of embodiments of this application or a related art more clearly, the following briefly introduces accompanying drawings required for describing the embodiments or the related art. Apparently, the accompanying drawings in the following description show only some embodiments of this application, and a person of ordinary skill in the art may still derive other accompanying drawings from these accompanying drawings without creative efforts. In the accompanying drawings:

FIG. 1 illustrates a block diagram of a system for detecting an anomaly of a physiological parameter detection apparatus, in accordance with some embodiments of the present disclosure.

FIG. 2 illustrates a block diagram of the anomaly detection device showing one or more modules associated with the operations of the anomaly detection device, in accordance with some embodiments.

FIG. 3 is a schematic flowchart of a method of detecting an anomaly of a physiological parameter detection apparatus, according to an embodiment of this application.

FIG. 4 is a schematic diagram of a working circuit of a physiological parameter detection apparatus in an application scenario according to an embodiment of this application.

FIG. 5 is a schematic diagram of a logical framework of anomaly type detection of a physiological parameter detection apparatus according to an embodiment of this application.

FIG. 6 is a schematic diagram of a method for detecting an anomaly in a human vital sign signal in a case according to an embodiment of this application; and

FIG. 7 is another schematic diagram illustrating a flow chart that integrates the logical framework of anomaly type detection and the method for detecting an anomaly in a human vital sign signal.

### DETAILED DESCRIPTION

To explain the overall concept of this application more clearly, the following describes in detail by using examples with reference to the accompanying drawings of the specification.

To understand the foregoing objectives, features, and advantages of this application more clearly, the following further describes this application in detail with reference to the accompanying drawings and specific implementations. It is to be noted that embodiments of this application and features in the embodiments may be combined without a conflict.

In the following description, many specific details are described to make this application fully understood. However, this application may alternatively be implemented in other manners different from those described herein. Therefore, the protection scope of this application is not limited to the specific embodiments disclosed below.

In addition, in the description of this application, it is to be understood that terms “first” and “second” are merely used for the purpose of description, and cannot be understood as indicating or implying relative importance or implicitly specifying the number of indicated technical features. Therefore, a feature defined by “first” or “second” may explicitly or implicitly include one or more features. In the descriptions of this application, “a plurality of” means two or more, unless otherwise definitely and specifically limited.

In this application, unless explicitly specified and defined otherwise, terms “install”, “interconnect”, “connect”, “fix”, and the like are to be understood in a broad sense, for example, may be a fixed connection, a detachable connection, or an integrated formation, may be a mechanical connection or an electrical connection, may be communication, may be a direct connection or an indirect connection through an intermediate medium, or may be an internal communication between two elements or an interaction relationship between two elements. A person of ordinary skill in the art may understand the specific meanings of the foregoing terms in this application according to specific situations.

In this application, unless otherwise explicitly specified or defined, the first feature being located “above” or “below” the second feature may be the first feature being in direct contact with the second feature, or the first feature being in indirect contact with the second feature through an intermediate medium. In the descriptions of this specification, descriptions of a reference term such as “an embodiment”, “some embodiments”, “an example”, “a specific example”, or “some examples” means that a feature, structure, material, or characteristic that is described with reference to the embodiment or the example is included in at least one embodiment or example of this application. In this specification, schematic expressions of the foregoing terms do not necessarily refer to the same embodiment or example. Moreover, the specific features, the structures, the materials or the characteristics that are described may be combined in proper manners in any one or more embodiments or examples.

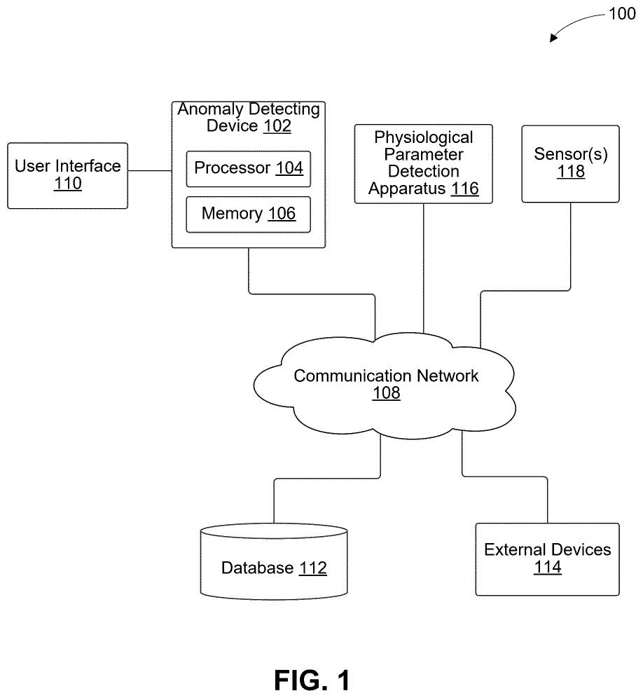

FIG. 1 illustrates a block diagram of a system 100 for detecting an anomaly of a physiological parameter detection apparatus 116, in accordance with some embodiments of the present disclosure. The physiological parameter detection apparatus 116 (alternatively “apparatus 116” hereinafter) may be configured to sense one or more physiological parameters of a subject and to generate a corresponding human vital sign signal based on the sensed parameter. The apparatus 116 may include signal conditioning circuitry, and processing components for acquiring and outputting physiological data. During operation, the apparatus 116 may produce working attributes indicative of its operational state, which are used for anomaly detection.

The system 100 may include an anomaly detection device 102 configured to detect an anomaly associated with the physiological parameter detection apparatus 116. The anomaly detection device 102 may have data processing capability. In particular, the anomaly detection device 102 may be implemented as a stand-alone device. Alternatively, the anomaly detection device 102 may be implemented as a desktop, a laptop, a notebook, a netbook, a tablet, a smartphone, a mobile phone, an application server, a web server, or the like.

The system 100 may further include one or more sensors 118 that may be configured to monitor one or more working attributes of the physiological parameter detection apparatus 116 during operation. The working attributes may include electrical, and/or thermal or mechanical parameters indicative of an operational state of the physiological parameter detection apparatus 116. The sensors 118 may generate sensor data that is supplied to the anomaly detection device 102.

The system 100 may further include a database 112 which may store data associated with the anomaly detection device 102. Additionally, the anomaly detection device 102 may be communicatively coupled to an external device 114 for sending and receiving various data. Examples of the external device 114 may include, but are not limited to, a remote server, digital devices, and a computer system. The anomaly detection device 102 may connect to the apparatus 116, the sensors 118, the database 112, and the external device 114 over a communication network 108. The communication network 108 may be a wired connection, for example via Universal Serial Bus (USB). A computing device, a smartphone, a mobile device, a laptop, a smartwatch, a personal digital assistant (PDA), an e-reader, and a tablet are all examples of the external device 114. For example, the communication network 108 may be a wireless network, a wired network, a cellular network, a Code Division Multiple Access (CDMA) network, a Global System for Mobile Communication (GSM) network, a Long-Term Evolution (LTE) network, a Universal Mobile Telecommunications System (UMTS) network, a Worldwide Interoperability for Microwave Access (WiMAX) network, a Dedicated Short-Range Communications (DSRC) network, a local area network, a wide area network, the Internet, satellite or any other appropriate network required for communication between the anomaly detection device 102, the apparatus 116, the sensors 118, the database 112, and the external device 114.

The anomaly detection device 102 may be configured to perform one or more operations, that may include receive, from the physiological parameter detection apparatus 116, a human vital sign signal indicative of a physiological parameter; determine, using a filtering model, an estimated vital sign signal at a current moment based on the human vital sign signal at a previous moment; generate a signal-state vector based on at least one of the human vital sign signal and the estimated vital sign signal; receive, from the at least one sensor 118, the working attribute of the physiological parameter detection apparatus; generate a hardware-state vector based on the at least one working attribute and a corresponding preset threshold; and determine whether an anomaly exists in the output of the physiological parameter detection apparatus based on the signal-state vector and the hardware-state vector.

To perform the above functionalities, the anomaly detection device 102 may include a processor 104 and a memory 106. The memory 106 may be communicatively coupled to the processor 104. The memory 106 stores a plurality of instructions, which upon execution by the processor 104, may cause the processor 104 to perform the above functionalities.

The system 100 may further implement a user interface 110. In an embodiment, the user interface 110 that may be implemented on a display, keypad, microphone, audio speakers, vibrating motor, LED lights, etc. The user interface may further display an output of the computation performed by the anomaly detection device 102. The user interface may either be integrated within the anomaly detection device 102 or may be implemented as a separate module.

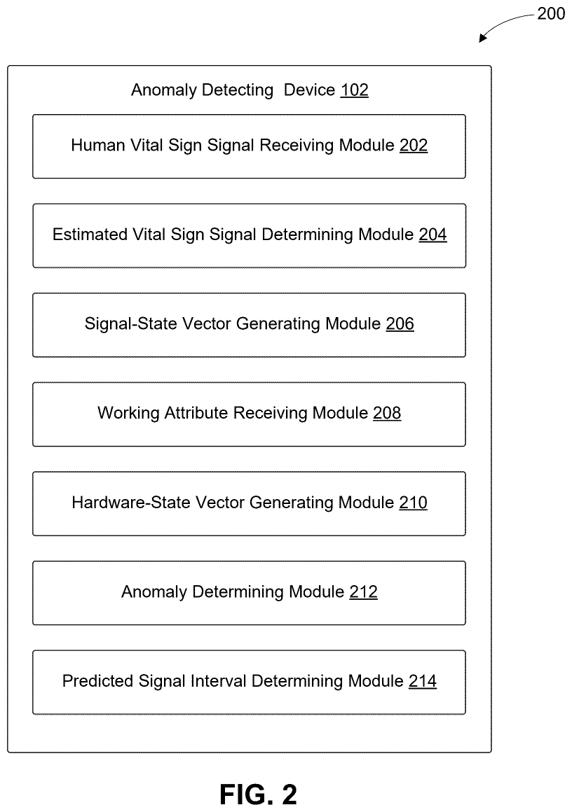

FIG. 2 illustrates a block diagram 200 of the anomaly detection device 102 showing one or more modules associated with the operations of the anomaly detection device 102, in accordance with some embodiments. In some embodiments, the anomaly detection device 102 implements a human vital sign signal receiving module 202, an estimated vital sign signal determining module 204, a signal-state vector generating module 206, a working attribute receiving module 208, a hardware-state vector generating module 210, an anomaly determining module 212, and a predicted signal interval determining module 214.

The human vital sign signal receiving module 202 may be configured to receive, from the physiological parameter detection apparatus 116, a human vital sign signal indicative of a physiological parameter. The human vital sign signal may be generated by the physiological parameter detection apparatus 116 during continuous or periodic monitoring of a subject and may represent a measured value associated with the physiological parameter.

The estimated vital sign signal determining module 204 may be configured to determine, using a filtering model, an estimated vital sign signal at a current moment based on the human vital sign signal at a previous moment. In some embodiments, the filtering model may include a state-space model describing temporal evolution of the human vital sign signal, and the estimated vital sign signal determining module 204 may perform prediction processing to generate an estimated value representative of an expected physiological state at the current moment. For example, the estimated vital sign signal determining module 204 may linearize a state equation of the filtering model to obtain a linear state equation, and may further obtain the estimated vital sign signal at the current moment based on an optimal estimation result of the human vital sign signal at the previous moment by applying the linear state equation, thereby enabling robust estimation of the vital sign signal under conditions of noise, disturbance, or signal uncertainty.

The signal-state vector generating module 206 may be configured to generate a signal-state vector based on at least one of the human vital sign signal and the estimated vital sign signal. The signal-state vector, for example, may represent a discrete or structured characterization of signal behavior at a given moment, and may be used to indicate whether the human vital sign signal exhibits abnormal amplitude variation, abnormal rate of change, or other signal-level irregularities relative to expected physiological behavior.

The preset signal value may include a first signal value, a second signal value, and a third signal value, each corresponding to a predefined signal constraint or variation threshold.

Before generating the signal-state vector, the signal-state vector generating module 206 may preprocess the human vital sign signal based on the first signal value, the second signal value, and the third signal value, to obtain a pre-processed vital sign update signal. Correspondingly, the signal-state vector generating module 206 may generate the signal-state vector further based on the vital sign update signal. The preprocessing may be performed to suppress excessive signal excursions or abrupt variations that are unlikely to represent true physiological changes.

For example, preprocessing the human vital sign signal may include updating the human vital sign signal to the first signal value if the human vital sign signal is greater than the first signal value; updating the human vital sign signal to a sum value of the human vital sign signal at the previous moment and the second signal value if the human vital sign signal is greater than the human vital sign signal at the previous moment and a difference is greater than the second signal value; and updating the human vital sign signal to a difference between the human vital sign signal at the previous moment and the third signal value if the human vital sign signal is less than the human vital sign signal at the previous moment and an absolute value of the difference is greater than the third signal value.

In some embodiments, the signal-state vector generating module 206 may generate the signal-state vector by: determining a state value corresponding to the vital sign update signal as a first preset value if the human vital sign signal is greater than or equal to the first signal value or if the vital sign update signal is 0; determining the state value corresponding to the vital sign update signal as the first preset value if the human vital sign signal is greater than the human vital sign signal at the previous moment and an absolute value of a difference is greater than or equal to the second signal value; determining the state value corresponding to the vital sign update signal as the first preset value if the human vital sign signal is less than the human vital sign signal at the previous moment and the absolute value of the difference is greater than or equal to the third signal value; determining the state value corresponding to the vital sign update signal as a second preset value if the human vital sign signal is not less than the human vital sign signal at the previous moment, or the absolute value of the difference is less than the third signal value; and generating the signal-state vector based on the state value of each vital sign update signal.

In some embodiments, the signal-state vector generating module 206 may generate the signal-state vector by: determining a state value corresponding to the estimated vital sign signal as a second preset value if the estimated vital sign signal falls within the predicted signal interval; determining the state value corresponding to the estimated vital sign signal as a first preset value if the estimated vital sign signal does not fall within the predicted signal interval; and generating the signal-state vector based on the state value of each estimated vital sign signal.

In some embodiments, the signal-state vector generating module 206 may generate the signal-state vector by: determining a state value corresponding to the vital sign update signal as a first preset value if the vital sign update signal is different from the human vital sign signal, or if the human vital sign signal is zero; determining the state value corresponding to the vital sign update signal as a second preset value if the vital sign update signal is equal to the human vital sign signal; and generating the signal-state vector based on the state value corresponding to each vital sign update signal.

The working attribute receiving module 208 may be configured to acquire at least one working attribute of the physiological parameter detection apparatus 116. The at least one working attribute may include a real-time working voltage and a real-time working current of the physiological parameter detection apparatus. The real-time working voltage may include a working electrode voltage, a reference electrode voltage, and a counter electrode voltage.

In some embodiments, prior to the obtaining the real-time working voltage and a real-time working current of the physiological parameter detection apparatus, the working attribute receiving module 208 may collect an initial voltage of the physiological parameter detection apparatus according to a preset sampling period; calculate an average voltage, a standard deviation, and a range value that correspond to the initial voltage, and determining whether the average voltage, the standard deviation, and the range value are all within corresponding preset thresholds; and if the average voltage, the standard deviation, and the range value are all within the corresponding preset thresholds, obtain the real-time working voltage and the real-time working current of the physiological parameter detection apparatus. The working attribute receiving module 208 may further determine that the real-time working current is an invalid signal if the voltage determination result or the current determination result is an abnormality.

The hardware-state vector generating module 210 may be configured to generate a hardware-state vector based on the at least one working attribute and a corresponding preset threshold.

In some embodiments, the predicted signal interval determining module 214 may be configured to determine, through a constraint model, a predicted signal interval at the current moment based on a mean and a standard deviation of the human vital sign signal within a preset time window. Generating the signal-state vector may include determining whether the estimated vital sign signal falls within the predicted signal interval.

In some embodiments, in order to determine the predicted signal interval, the predicted signal interval determining module 214 may calculate a signal standard deviation and a signal mean based on the human vital sign signal within the preset time window; and input the signal standard deviation, the signal mean, the human vital sign signal at the previous moment, and the estimated vital sign signal to the constraint model, and determining, by the constraint model, the predicted signal interval at the current moment.

The predicted signal interval at the current moment may include a maximum critical value and a minimum critical value. A calculation formula of the maximum critical value is:

$${upLimit\_CgmSignal}_{i} = G ( {Pro\_CgmSignal}_{i - 1} , Ave\_Pro {\_CgmSignal}_{N} , {\overline{x}}_{k + 1 \vert k} ) + γ * Std\_Pro {\_CgmSignal}_{N}$$

A calculation formula of the minimum critical value is:

$${LowLimit\_CgmSignal}_{i} = G ( {Pro\_CgmSignal}_{i - 1} , Ave\_Pro {\_CgmSignal}_{N} , {\overline{x}}_{k + 1 \vert k} ) - β * Std\_Pro {\_CgmSignal}_{N}$$

wherein,
Pro_CgmSignalᵢ₋₁ represents the human vital sign signal at the previous moment;
Ave_Pro_CgmSignal_{N} represents the signal mean;

$${Std}_{{Pro}_{{CgmSignal}_{N}}}$$

represents the signal standard deviation; and
γ and β represent preset coefficients.

The anomaly determining module 212 may be configured to determine whether an anomaly exists in the output of the physiological parameter detection apparatus 116 based on the signal-state vector and the hardware-state vector.

In some embodiments, determining whether the anomaly exists may additionally include evaluating a state value of the signal-state vector corresponding to the current moment, and determining that an anomaly exists when the state value is equal to the second state value.

The preset threshold corresponding to the real-time working voltage may include a first preset voltage threshold and a second preset voltage threshold. The anomaly determining module 212 may further compare the working electrode voltage with the first preset voltage threshold, and comparing the reference electrode voltage with the second preset voltage threshold, to determine a first voltage determination result; determine a second voltage determination result based on the working electrode voltage, the reference electrode voltage, and the counter electrode voltage; and determine the anomaly in the output of the physiological parameter detection apparatus, when there is an abnormal result in the first voltage determination result or the second voltage determination result.

In some embodiments, a first preset threshold corresponding to the real-time working voltage may include a first preset voltage difference threshold and a second preset voltage difference threshold. As such, determining the second voltage determination result may include determining a first voltage difference between the working electrode voltage and the reference electrode voltage; determining a second voltage difference between the working electrode voltage and the counter electrode voltage; and determining that the second voltage determination result indicates an abnormality when the first voltage difference is greater than the first preset voltage difference threshold or the second voltage difference is greater than the second preset voltage difference threshold.

The anomaly determining module 212 may be further configured to compare the real-time working current with a second preset threshold to determine a current determination result. Accordingly, the anomaly determining module 212 may obtain a first scope value corresponding to detection sensitivity of the physiological parameter detection apparatus, and obtaining a second scope value corresponding to a to-be-detected physiological parameter; determine a threshold interval of the real-time working current according to the first scope value and the second scope value, and determining the real-time working current based on the threshold interval, to obtain a first current determination result; perform signal filtering processing on the real-time working current, and determining a second current determination result according to the processed real-time working current and a preset current threshold; perform low-pass filtering processing on the real-time working current to obtain a baseline of the real-time working current, and determining a third current determination result based on drift data of the baseline and a corresponding preset drift data threshold; and determine that the current determination result indicates an abnormality if there is an abnormal result in the first current determination result, the second current determination result, or the third current determination result.

Further, the anomaly determining module 212 may determine an anomaly type for the anomaly, the wherein the anomaly type comprises a soft anomaly and a hard anomaly. The anomaly determining module 212, as such, may further determine that the anomaly type is the hard anomaly if at least one of the first voltage determination result or the second voltage determination result is an abnormality; determine that the anomaly type is the hard anomaly if the third current determination result is an abnormality; detect whether the abnormality is corrected within a preset time step if the first current determination result or the second current determination result is an abnormality; determine that the anomaly type is the soft anomaly if the abnormality is corrected within the preset time step; and determine that the anomaly type is the hard anomaly if the abnormality is not corrected within the preset time step.

In some embodiments, the anomaly determining module 212 may further count a number of times the physiological parameter detection apparatus 116 has a hard anomaly; and cause the physiological parameter detection apparatus 116 to output an alarm signal if the counted number of times exceeds a preset number threshold.

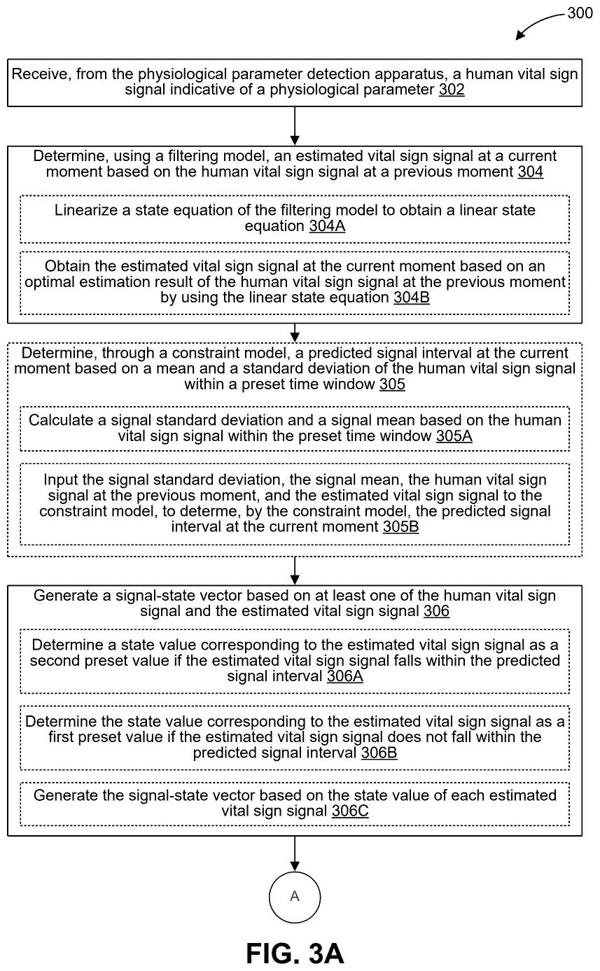

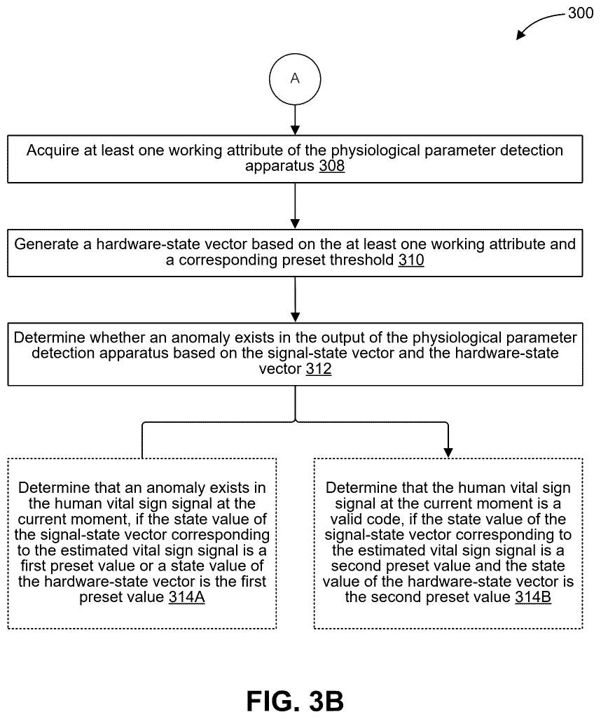

An embodiment of this application provides a method of detecting an anomaly of a physiological parameter detection apparatus 116, in accordance with some embodiments. FIG. 3 is a schematic flowchart of a method 300 of detecting an anomaly of a physiological parameter detection apparatus 116, according to an embodiment of this application. As shown in FIG. 1, the method includes:

At step 302, a human vital sign signal indicative of a physiological parameter is received from the physiological parameter detection apparatus. The human vital sign signal represents an output generated by the apparatus during operation and is used as a primary input for subsequent signal-level analysis.

At step 304, an estimated vital sign signal at a current moment is determined using a filtering model based on the human vital sign signal at a previous moment. As such, step 304 may correspond to temporal estimation of the expected physiological signal behavior and assist in mitigating the effects of noise, transient artifacts, and irregular fluctuations.

In some embodiments, the filtering model is a state-space model that describes temporal evolution of the human vital sign signal. The filtering model may include a state equation and an observation equation, both of which may be nonlinear in practical physiological monitoring scenarios.

The step 304 may include a sub-step 304A at which a state equation of the filtering model may be linearized to obtain a linear state equation. Linearization may be performed by applying a first-order Taylor series expansion around a prior estimated state, while higher-order terms are neglected. The resulting linear state equation enables use of a linear filtering framework.

At sub-step 304Ab, based on the linear state equation, an optimal estimation result of the human vital sign signal at the previous moment may be used to obtain the estimated vital sign signal at the current moment. In some embodiments, the filtering model may be implemented using a Kalman filter, an extended Kalman filter, an unscented Kalman filter, or another recursive estimation algorithm suitable for time-series physiological data.

The estimated vital sign signal may represent an expected value of the physiological parameter at the current moment under normal operating conditions and physiological continuity assumptions.

In some embodiments, at step 305, a predicted signal interval at the current moment may be determined through a constraint model. The predicted signal interval represents an expected range within which a valid estimated vital sign signal is anticipated to fall, based on historical signal statistics.

To this end, at sub-step 305A, a signal mean and a signal standard deviation may be calculated based on the human vital sign signal within a preset time window. The preset time window may include a fixed number of previous signal samples or may correspond to a predefined temporal duration. The signal mean may represent a central tendency of the physiological signal over the preset time window, while the signal standard deviation represents the degree of variability or dispersion of the signal values within the same window.

At sub-step 305B, the signal standard deviation, the signal mean, the human vital sign signal at the previous moment, and the estimated vital sign signal may be input into the constraint model to determine the predicted signal interval at the current moment.

In some embodiments, the predicted signal interval includes a maximum critical value and a minimum critical value. The maximum critical value may be calculated using a function that incorporates the human vital sign signal at the previous moment, the signal mean, and the estimated vital sign signal, together with a positive coefficient multiplied by the signal standard deviation. Similarly, the minimum critical value may be calculated using the same function with a negative coefficient multiplied by the signal standard deviation.

The constraint model, therefore, provides an adaptive signal envelope that reflects both historical variability and short-term signal dynamics, allowing the system to distinguish expected physiological variations from abnormal deviations.

A blood glucose value is used as an example, use or interference exists in an actual continuous glucose monitor (CGM) signal system. Consequently, an acquired blood glucose value signal is nonlinear. In a prediction process of the filtering model, a nonlinear system may be approximately regarded as a linear system, thereby obtaining an accurate prediction result.

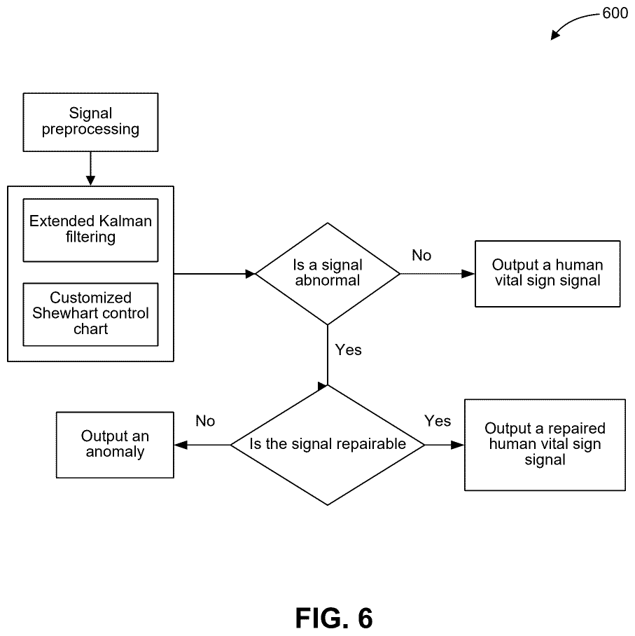

Based on this, as shown in FIG. 6, in the nonlinear system 600, the filtering model performs Taylor series expansion around a filtering value on a state equation and an observation equation that are nonlinearized, and second-order and higher-order terms are deleted, thereby linearizing the state equation. The nonlinear system is approximated as a linear system, and then a standard Kalman filtering algorithm is used to estimate the signal vector of the human vital sign signal.

For elaboration, a standard Kalman filtering technology is usually applicable to making optimal estimation on a target state under a condition of a linear Gaussian model. Its state equation is: xₖ₊₁=f(x)+Wₖ, and its observation equation is: yₖ=h(xₖ)+vₖ, where the state equation f and the observation equation h are nonlinear functions; k represents discrete time; process noise Wₖ₋₁ and observation noise vₖ are respectively irrelevant and satisfy a Gaussian distribution with covariances of Qₖ and Rₖ.

It is assumed that a state estimate x̅_{k|k} and an estimated variance P_{k|k} at a k moment are known, and Taylor series expansion is performed on a state equation and an observation equation. After higher-order items are ignored, the state equation is xₖ₊₁≈f(x̅_{k|k})+Fₖ(xₖ−x̅_{k|k}) and the observation equation is yₖ₊₁≈h(x̅_{k+1|k})+Hₖ₊₁(xₖ₊₁−x̅_{k+1|k}). Where Fₖ and Hₖ are respectively jacobian matrixes of the state equation f and the observation equation h at x̅ₖ.

In this case, a linearized filtering model is used for prediction. The filtering model includes:

$${\overline{x}}_{k + 1 \vert k} = f ( {\overline{x}}_{k \vert k} ) and {P}_{k + 1 \vert k} = {F}_{k} {P}_{k} {F}_{k}^{T} + {Q}_{k} .$$

Where x̅_{k+1|k} represents prediction on an estimated feature signal xₖ₊₁ at a (k+1)^{th} moment based on information at the k moment; P_{k+1|k} represents a predicted covariance matrix; and Qₖ represents a covariance matrix of the process noise.

It should be noted that when a new inputted signal y_{k+1|k} is determined as a valid signal, the state estimate needs to be updated to obtain an optimal estimation result corresponding to the inputted signal for application to signal detection at a next moment. A determining process of the corresponding optimal estimation result is as follows:

$${K}_{k + 1} = {P}_{k + 1 \vert k} {{H}_{k + 1}^{T} ( {H}_{k + 1} {P}_{k + 1 \vert k} {H}_{k + 1}^{T} + {R}_{k + 1} )}^{- 1} ;$$

$${\overline{x}}_{k + 1 \vert k + 1} = {\overline{x}}_{k + 1 \vert k} + {K}_{k + 1} ( {y}_{k + 1} - h ( {\overline{x}}_{k + 1 \vert k} ) ) ;$$

$${P}_{k + 1 \vert k + 1} = ( I - {K}_{k + 1} {H}_{k + 1} ) {P}_{k + 1 \vert k} ,$$

where Kₖ₊₁ represents a Kalman gain; x̅_{k+1|k+1} represents an updated state estimate; P_{k+1|k+1} represents an updated covariance matrix; and Rₖ₊₁ represents a covariance matrix of the observation noise.

When prediction is performed through the filtering model, the state equation f and the observation equation h that are included in the filtering model are first determined, and initial values of parameters of the filtering model are set. Setting initial values of parameters required in an estimation process mainly includes distribution of initial states, an initial distribution of a covariance matrix of the distribution of the initial states, a process noise covariance matrix, and a measurement noise covariance matrix, and the like.

By using the state equation, a first predicted value of a human vital sign signal at a current moment (the (k+1)^{th} moment) is obtained based on an optimal estimation result of a human vital sign signal at a previous moment (the k^{th} moment).

A linearized state equation is obtained by calculating a jacobian matrix of a nonlinear state equation, and a second predicted value of an error covariance matrix at the current moment (the (k+1)^{th} moment) based on the linearized state equation and an error covariance matrix at the previous moment (the k^{th} moment).

A filter gain is obtained based on the first predicted value and the second predicted value. The filter gain is used for measuring a weight of old and new information. An optimal estimation result of the human vital sign signal at the current moment (the (k+1)^{th} moment) and a posteriori estimate of the error covariance matrix are obtained based on the filter gain and a measured value of the human vital sign signal at the previous moment (the k^{th} moment). Finally, the process is repeated until a final moment is calculated.

Certainly, in addition to the standard Kalman filtering algorithm, another filtering algorithm may be used, for example, an extended Kalman filtering algorithm, an unscented Kalman filtering algorithm, or a particle filtering algorithm. In some filtering algorithms, vector estimation may be performed without linearizing the state equation, thereby improving estimation accuracy and increasing algorithm complexity. Selection may be performed based on an actual case.

Certainly, in addition to the filtering algorithm, for different scenarios, a time series analysis algorithm such as an autoregressive moving average model or an autoregressive integrated moving average model, or a neural network algorithm such as a recursive neural network or a long short-term memory network, may be used to estimate a signal vector based on an actual need.

The filtering model is mainly used for estimating the human vital sign signal, and the constraint model is mainly used for constraining the predicted signal interval, so as to facilitate determining, in a subsequent signal processing process, whether an anomaly exists in the inputted signal.

Specifically, as shown in FIG. 6, constructing a constraint model by using a Shewhart control chart is used as an example for explanation.

First, an appropriate time window value N is selected. For a human vital sign signal within a preset time window N, a mean and a standard deviation that correspond to the human vital sign signal are determined.

An mean Ave_Pro_CgmSignal_{N} corresponding to [Pro_CgmSignal_{i-N-1}, Pro_CgmSignalᵢ₋₁] time window N is calculated based on

$$Ave\_Pro {\_CgmSignal}_{N} = \frac{1}{N} {\sum }_{i = 1}^{N} ( {Pro\_CgmSignal}_{i} ) .$$

A standard deviation Std_Pro_CgmSignal_{N} corresponding to Pro_CgmSignal_{i-N-1}, Pro_CgmSignalᵢ₋₁] time window N is calculated based on

$$Std\_Pro {\_CgmSignal}_{N} = \sqrt[2]{\frac{{\sum }_{i = 1}^{N} {( {Pro\_CgmSignal}_{i} - Ave\_Pro {\_CgmSignal}_{N} )}^{2}}{N}} .$$

At least one of the mean and the standard deviation and the human vital sign signal at the previous moment are inputted to the constraint model based on the Shewhart control chart, and a maximum critical value and a minimum critical value of the predicted signal interval of the human vital sign signal at the current moment are outputted.

Specifically, positive compensation is performed on the human vital sign signal at the previous moment by using a third preset coefficient and the standard deviation, to obtain the maximum critical value of the predicted signal interval at the current moment. In addition, negative compensation is performed on the human vital sign signal at the previous moment by using a fourth preset coefficient and the standard deviation, to obtain the minimum critical value of the predicted signal interval at the current moment. Values of the third preset coefficient and the fourth preset coefficient are positively correlated with the human vital sign signal at the previous moment.

For example, in conjunction with information of the human vital sign signal Pro_CgmSignalᵢ₋₁ at the previous moment and the estimated vital sign signal x̅_{k+1|k} at the current moment, the mean Ave_Pro_CgmSignal_{N} and the standard deviation Std_Pro_CgmSignal_{N} are inputted to a customized Shewhart control chart constraint model to obtain the predicted signal interval of the human vital sign signal CgmSignalᵢ at the current moment.

The customized Shewhart control chart constraint model may be:

$${upLimit\_CgmSignal}_{i} , {lowLimit\_CgmSignal}_{i} = f ( Ave\_Pro {\_CgmSignal}_{N} , {\overline{x}}_{k + 1 \vert k} , Std\_Pro {\_CgmSignal}_{N} , {Pro\_CgmSignal}_{i - 1} )$$

where the maximum critical value is:

$${upLimit\_CgmSignal}_{i} = G ( {Pro\_CgmSignal}_{i - 1} , Ave\_Pro {\_CgmSignal}_{N} , {\overline{x}}_{k + 1 \vert k} ) + γ * Std\_Pro {\_CgmSignal}_{N} ,$$

the minimum critical value is:

$${LowLimit\_CgmSignal}_{i} = G ( {Pro\_CgmSignal}_{i - 1} , Ave\_Pro {\_CgmSignal}_{N} , {\overline{x}}_{k + 1 \vert k} ) - β * Std\_Pro {\_CgmSignal}_{N} ,$$

where γ and β respectively represent the third preset coefficient and the fourth preset coefficient, coefficient values of which need to be determined based on Pro_CgmSignalᵢ₋₁. When Pro_CgmSignalᵢ₋₁ is relatively small, the coefficient values of γ and β are decreased, and when Pro_CgmSignalᵢ₋₁ is relatively large, the coefficient values of γ and β are increased, thus implementing a positive correlation. The γ and β coefficients are non-zero positive values. Values of the third preset coefficient and the fourth preset coefficient are positively correlated to the value of the human vital sign signal at the previous moment and can implement corresponding positive compensation and negative compensation, and are not limited to the manners in the above formulas.

Certainly, in addition to a Shewhart control chart, a similar function may be implemented by using a manner such as a cusum control chart, a control chart of exponential weighted moving average, or a single value-moving R-control chart, to determine an upper track and a lower track of the signal interval.

Or, the maximum critical value and the minimum critical value of the signal interval may be determined in a manner such as an autoregressive model, a neural network, or support vector regression based on a need.

At step 306, the anomaly detecting logic (e.g., signal-state vector generating module 206 executed by processor 104) may generate a signal-state vector for the current moment by evaluating at least one of: (i) the received human vital sign signal (e.g., an instantaneous glucose current-derived value, ECG-derived metric, SpO₂-derived value, etc.), and/or (ii) the estimated vital sign signal produced by the filtering model at the current moment. In particular, at step 306, the signal-state vector may be generated as a time-indexed sequence of discrete state values, each state value representing whether a corresponding estimated vital sign signal is deemed consistent with a predicted signal interval (as determined by the constraint model). The resulting signal-state vector may later be combined with a hardware-state vector generated from one or more working attributes (e.g., voltage/current attributes) to determine whether an anomaly exists in the output of the physiological parameter detection apparatus.

At sub-step 306A, a state value corresponding to the estimated vital sign signal may be determined as a second preset value (e.g., a “normal” value such as 1) when the estimated vital sign signal at the current moment falls within the predicted signal interval for the same moment. In some implementations, the predicted signal interval may be expressed as a bounded range having an upper critical value and a lower critical value, and the “falls within” condition may be satisfied when the estimated vital sign signal is greater than or equal to the lower critical value and less than or equal to the upper critical value. By assigning the second preset value under this condition, the system 100 may record (i.e., within the signal-state vector) that the estimated vital sign signal is statistically and temporally plausible given (a) the recent behavior of the human vital sign signal within a preset time window (e.g., via mean/standard deviation), and (b) the filtering-model-derived expectation at the current moment.

At sub-step 306B, the state value corresponding to the estimated vital sign signal may be determined as a first preset value (e.g., an “abnormal” value such as 0) when the estimated vital sign signal at the current moment does not fall within the predicted signal interval. This “does not fall within” condition may occur, for example, when the estimated vital sign signal exceeds the upper critical value (indicating an unexpected upward deviation) or drops below the lower critical value (indicating an unexpected downward deviation), relative to the constrained prediction band derived from the recent time window statistics and the constraint model. In some embodiments, sub-step 306B may provide for an evidence that the present output of the physiological parameter detection apparatus may be unreliable, corrupted, or otherwise anomalous, particularly when combined with contemporaneous indications of abnormal hardware behavior (e.g., voltage/current determination results reflected in a hardware-state vector). As such, assigning the first preset value may enable downstream anomaly decision logic to apply simple, deterministic rules (e.g., OR/AND logic with the hardware-state vector, persistence checks, or alarm-threshold checks) while still capturing the essence of a statistically significant deviation at the current moment.

At sub-step 306C, the signal-state vector may be generated (or updated) based on the state value of each estimated vital sign signal. For example, as each new current-moment estimated vital sign signal is produced by the filtering model, the system may append the corresponding state value (first preset value or second preset value) to an ordered vector structure indexed by time.

In one implementation, the signal-state vector may be a binary vector (SV=[sv1, sv2, . . . , svi]), where each element svᵢ corresponds to the ith moment and equals the second preset value when the estimated vital sign signal is inside the predicted signal interval (sub-step 306A) and equals the first preset value when it is outside the predicted signal interval (sub-step 306B). In another implementation, the signal-state vector may be stored as a rolling buffer spanning a configurable number of most recent moments, thereby supporting persistence-based anomaly decisions (e.g., distinguishing transient deviations from sustained anomalies). The signal-state vector generated in sub-step 306C may then be provided to subsequent steps that determine whether an anomaly exists based on joint consideration of (i) the signal-state vector and (ii) a hardware-state vector derived from one or more working attributes of the physiological parameter detection apparatus (e.g., real-time working voltage/current and corresponding thresholds).

If the estimated vital sign signal falls within the predicted signal interval, a state value corresponding to the estimated vital sign signal is determined as a second preset value; if the estimated vital sign signal does not fall within the predicted signal interval, the state value corresponding to the estimated vital sign signal is determined as a first preset value; and a second state vector is generated based on the state value of each estimated vital sign signal.

Specifically, a priori estimate value x̅_{k|k-1} at the current moment is calculated based on the filtering model by using an optimal state estimate value x̅ₖ₋₁ at the previous moment and a measurement signal xₖ at the current moment, and whether x̅_{k|k-1} falls within the predicted signal interval [LowLimit_CgmSignalᵢ, upLimit_CgmSignalᵢ] is determined; if yes, a state value CgmInGapᵢ of the second state vector is recorded as the second preset value of 1; and if no, the state value is recorded as the first preset value of 0. Thus, the second state vector [CgmInGap₁, CgmInGap₂, CgmInGap₃ . . . ] is generated.

In some embodiments, the human vital sign signal may refer to a related signal that is acquired through a corresponding device and a sensor and can represent a human vital sign of a user. For example, the human vital sign signal may be a blood glucose signal of a user that is acquired by implanting a continuous glucose monitor (CGM) sensor under the skin of the user, or an electrocardiosignal of the user that is acquired by an electrocardiograph, or a blood pressure signal, a blood oxygen signal, or the like of the user that is detected through a sphygmomanometer or an oximeter.

Causes of an anomaly may be roughly classified into the following several types: a low current phenomenon caused by a device fault, a current saturation phenomenon caused by a short circuit between electrodes of a device, a current signal fluctuation phenomenon generated by a device or in a use process, a severe vibration phenomenon of a current signal that is generated by an abnormal electrode or in a use process, a continuously decreasing phenomenon of a current that is caused by attenuation of performance of an electrode, and the like.

A blood glucose signal is used as an example. A process of acquiring a signal through a CGM is easily affected by problems such as noise, a sensor performance drift, breakage of a screen-printed electrode, a calibration error, and wearing compression. As a result, a current obtained on a working electrode is not a Faradic current generated by an actual reaction between glucose and an enzyme. By performing anomaly detection (i.e. ErrorCode detection), a valid and normal working current I_{w} is identified. This improves accuracy and reliability of monitoring blood glucose by the CGM, reduces an error rate, and facilitates analyzing and locating an anomaly.

Before step 306 is performed, initialization needs to be performed in advance, and a signal interval and a preset signal value that correspond to a human vital sign signal are set for the human vital sign signal. The signal interval mainly refers to intervals in which the human vital sign signal is a normal signal, intervals in which the human vital sign signal is an apparently abnormal signal, and the like. The signal interval may be further divided into a plurality of sub-intervals based on a need, or different signal interval division manners may be set based on different scenarios. The preset signal value includes a first signal value, a second signal value, and a third signal value, and are respectively critical values of corresponding signal intervals.

Specifically, for a signal value of the human vital sign signal, a first signal interval corresponding to the signal value is generated based on a preset conventional value interval and sensitivity of a data detection sensor. The conventional value interval refers to an interval in which the human vital sign signal falls when a human body is normal, and may be obtained based on a corresponding medical standard or big data analysis.

In context of blood glucose value of a human body used as an example, the conventional value interval is expressed by [Gluₘᵢₙ, Gluₘₐₓ], and a range of the conventional value interval of the signal value is preferably [3 mmol/L, 30 mmol/L]. This range is merely an exemplary scope in a conventional scenario. During actual execution, it may be correspondingly changed based on a change in the scenario (for example, a change based on an external condition such as the age of a user or a district of a user). Unless otherwise specified, the ranges exemplified in this embodiment of this application are all exemplary ranges, and do not represent fixed values of corresponding intervals or values. Details will not be elaborated subsequently.

The sensitivity of the data detection sensor is represented by Ks. In this case, the first signal interval may be [Gluₘᵢₙ*Ks, Gluₘₐₓ*Ks], which is a range of the working current I_{w}.

Similarly, for a change value of the human vital sign signal, a second signal interval corresponding to the change value is generated based on the preset conventional value interval. There may be a plurality of second signal intervals which at least respectively correspond to a rising value and a falling value.

Further, in context of the blood glucose value of a human body used as an example, a falling value of the blood glucose of the human body usually does not exceed 4 mmol/L to 5 mmol/L every hour. In this case, the falling value can be set to 6 mmol/L, and a conventional value interval [Glu_down_driftₘᵢₙ, Glu_down_driftₘₐₓ] of the falling value may be set to [0, 0.1 mmol/L/min]. Similarly, a rising value of the blood glucose of the human body can usually reach 0.2 mmol/L/min to 0.5 mmol/L/min. A conventional value interval [Glu_up_driftₘᵢₙ, Glu_up_driftₘₐₓ] of the rising value can be set to [0, 0.5 mmol/L/min]. In this case, for the rising value and the falling value, the conventional value intervals corresponding to the rising value and the falling value may be directly set to second signal intervals corresponding to the rising value and the falling value.

By using only the conventional value interval, it may be difficult to describe the impact of other factors in a real scenario during actual measurement. Therefore, for the first signal interval, a left endpoint is corrected to a fixed value less than a current value, and a right endpoint is corrected to be a variable value that is greater than the current value and is obtained by using the current value and a first coefficient. For example, the left endpoint of the first signal interval is corrected to 0, and the right endpoint is corrected to Gluₘₐₓ*Ks*(1+α), where a represents a non-zero positive value, which is preferably 20%, thereby expanding the range of the right endpoint. Certainly, the right endpoint may alternatively be represented in another manner, as long as the range of the right endpoint can be expanded, thereby expanding the first signal interval and improving robustness. Correspondingly, the first signal value is set to Gluₘₐₓ*Ks*(1+α).

Similarly, for the second signal interval, the right endpoint is corrected to be a variable value that is greater than the current value and is obtained by using the current value and a second coefficient. For example, the right endpoint of the second signal interval of the falling value is corrected to Glu_down_driftₘₐₓ*(1+β). When Glu_down_driftₘₐₓ=0.1 mmol/L/min, the second signal interval of the falling value is [0, 0.1*(1+β) mmol/L/min]. The right endpoint of the second signal interval of the rising value is corrected to Glu_up_driftₘₐₓ*(1+λ). When Glu_up_driftₘₐₓ=0.5 mmol/L/min, the second signal interval of the rising value is [0, 0.5*(1+λ) mmol/L/min]. Where both β and i are non-zero positive values and are preferably 30%, thereby expanding the range of the right endpoint. The right endpoint may alternatively be represented in another manner, as long as the range of the right endpoint can be expanded, thereby expanding the second signal interval and improving robustness. Correspondingly, the second signal value is set to Glu_up_driftₘₐₓ*(1+λ), and the third signal value is set to Glu_down_driftₘₐₓ*(1+β).

A corresponding human vital sign signal is obtained after initialization. In this case, an initialized signal interval and an initialized preset signal value are determined for the human vital sign signal. For different human vital sign signals, different signal intervals and preset signal values may be respectively initialized.

As shown in FIG. 6, the human vital sign signal is preprocessed based on the preset signal value, so as to update the human vital sign signal. An objective of preprocessing is to update a human vital sign signal with a corresponding value exceeding the preset signal value, to facilitate subsequent processing. A signal that has been preprocessed may be correspondingly marked, so as to determine, in a subsequent processing process based on the mark, whether the signal has been updated.

Specifically, an inputted human vital sign signal CgmSignalᵢ is obtained, where i represents an i^{th} moment. If a signal value of the human vital sign signal exceeds the first signal value, namely CgmSignalᵢ>Gluₘₐₓ*Ks*(1+α), the first signal value Gluₘₐₓ*KS*(1+α) is used as an update value of the human vital sign signal.

Based on the human vital sign signal, a change value ΔSignalᵢ of the human vital sign signal is obtained by using ΔSignalᵢ=CgmSignalᵢ−CgmSignalᵢ₋₁, and CgmSignalᵢ₋₁ represents a human vital sign signal at an (i−1)^{th} moment,
where the change value includes a rising value and a falling value. For the rising value, if the change value of the human vital sign signal exceeds the second signal value corresponding to the rising value, namely ΔSignalᵢ>Glu_up_driftₘₐₓ*(1+λ), the human vital sign signal CgmSignalᵢ is updated to CgmSignalᵢ₋₁+Glu_up_driftₘₐₓ*(1+λ). Certainly, this value cannot exceed the first signal value Gluₘₐₓ*KS*(1+α).

Similarly, for the falling value, if the change value of the human vital sign signal exceeds the third signal value corresponding to the falling value, namely ΔSignalᵢ<−Glu_down_driftₘₐₓ*(1+β), the human vital sign signal CgmSignalᵢ is updated to CgmSignalᵢ₋₁−Glu_down_driftₘₐₓ*(1+β). Certainly, this value needs to be greater than 0.

After the human vital sign signal is preprocessed to obtain a vital sign update signal, the vital sign update signal meets a requirement of the preset signal value. In this case, a first state vector may be generated based on a preprocessed human vital sign signal and the preset signal value.

The vital sign update signal is represented in a vector form, thus obtaining a signal vector. The first state vector mainly indicates whether the signal vector is updated because the signal vector exceeds the preset signal value.

Specifically, when the vital sign update signal CgmSignalᵢ≥Gluₘₐₓ*Ks*(1+α), or CgmSignalᵢ=0, or ΔSignalᵢ>Glu_up_driftₘₐₓ*(1+λ), or ΔSignalᵢ≤−Glu_down_driftₘₐₓ*(1+β), it indicates that the vital sign update signal is updated because the signal vector exceeds the preset signal value. In this case, a corresponding first state vector CgmStatusᵢ is recorded as a first preset value of 0, and may be recorded as a second preset value of 1 in other cases.

Two groups of vector data are obtained after the human vital sign signal is processed as above:

$$a set {Pro\_CgmSignal}_{n} = [ {Pro\_CgmSignal}_{1} , {Pro\_CgmSignal}_{2} … {Pro\_CgmSignal}_{n} ]$$

corresponding to the human vital sign signal, where Pro_CgmSignalᵢ refers to a preprocessed and updated vital sign update signal, and n represents a total number of moments; and

$$a set {CgmStatus}_{n} = [ {CgmStatus}_{1} , {CgmStatus}_{2} … {CgmStatus}_{n} ] corresponding to the first state vector .$$

In this case, the signal vector and the first state vector are used as inputs of a filtering model and a constraint model, to facilitate subsequent identification of an anomaly.

At step 308, at least one working attribute of the physiological parameter detection apparatus is acquired. The working attribute may represent an operational parameter of the apparatus 116 and may be independent of the human vital sign signal itself.

In some embodiments, the working attributes include a real-time working voltage and a real-time working current of the physiological parameter detection apparatus 116. The real-time working voltage may further include a working electrode voltage, a reference electrode voltage, and a counter electrode voltage, particularly in electrochemical sensing systems.

The working attributes may be acquired using dedicated sensors (e.g., sensors 118) configured to monitor electrical, thermal, or mechanical parameters of the apparatus during operation.

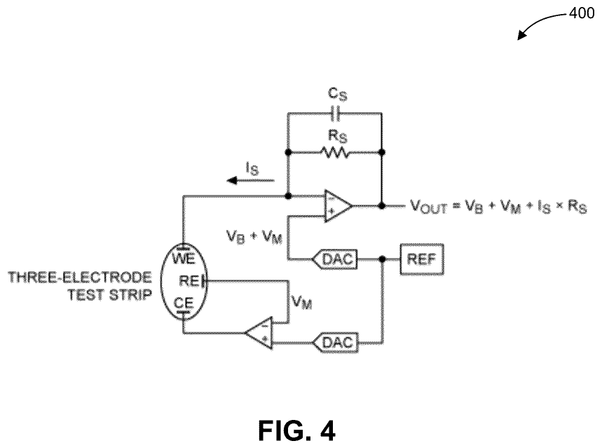

The physiological parameter detection apparatus 116 generally collects a physiological signal of a human body based on the sensors 118. Using a Continuous Glucose Monitor (CGM) shown in FIG. 4 as an example, when a sensor micro-needle is implanted into a body, an enzyme layer of a working electrode undergoes an electrochemical reaction with glucose in tissue liquid to generate a weak current (level nA). A circuit is formed between the working electrode and a counter electrode. Then, a current of the circuit is measured and a concentration of the glucose is calculated by using an algorithm. A stable voltage is provided for the working electrode for continuous and stable response, to ensure a stable oxidation reaction of the glucose and enzyme on the working electrode. The CGM fluctuates randomly when having a anomaly I_{w}. In this case, a blood glucose value measured I_{w} by the working electrode is not a real blood glucose value of the human body. Therefore, to quickly capture a change of a working signal of the apparatus, a timely information source is provided for anomaly detection, so that an anomaly can be detected in an early stage, and an invalid signal can be prevented from being continuously used. In this application, the real-time working voltage and the real-time working current of the physiological parameter detection apparatus 116 are obtained.

As shown in FIG. 2, in an embodiment, the real-time working voltage includes a working electrode voltage WEVolᵢ, a reference electrode voltage REVolᵢ, and a counter electrode voltage CEVolᵢ. By monitoring a real-time working voltage and a real-time working current of a physiological parameter detection apparatus 116 in real time, a change of a working signal of the apparatus can be quickly captured, and a timely information source is provided for anomaly detection, so that an anomaly can be detected in an early stage, and continuity and stability of a device can be ensured for a physiological parameter detection apparatus 116 that needs to continuously operate, thereby improving reliability and accuracy of the physiological parameter detection apparatus 116.

In an embodiment, to ensure stable performance and an accurate measurement capability of a sensor electrode at an initialization stage, so as to ensure that a reliable real-time working current and real-time working voltage are obtained, before obtaining the real-time working voltage and the real-time working current of the physiological parameter detection apparatus 116, the method 300 further includes the following process.

First, an initial voltage of the physiological parameter detection apparatus 116 is collected according to a preset sampling period. For example, the preset sampling period is 0.5 hour, and if a sampling rate is 3 min/Point, initial voltages of 10 points may be obtained. In this case, an average voltage, a standard deviation, and a range value that correspond to the initial voltage may be calculated, thereby determining whether the average voltage, the standard deviation, and the range value are all within corresponding preset thresholds. If the average voltage, the standard deviation, and the range value are all within corresponding preset thresholds, it indicates that the physiological parameter detection apparatus 116 has a reliable measurement capability. In this case, the real-time working voltage and the real-time working current of the physiological parameter detection apparatus 116 can be obtained.

Specifically, first, an initial voltage of the physiological parameter detection apparatus 116 needs to be obtained after a sensor of the physiological parameter detection apparatus 116 completes a polarization process, so as to calculate, based on the following formula, the average voltage, the standard deviation, and the range value that correspond to the initial voltage. When the initial voltage includes the working electrode voltage and the reference electrode voltage, an average voltage of the working electrode voltage is:

$$WEAvr = \frac{1}{n} {\sum }_{i = 1}^{n} {WEVol}_{i} ,$$

where WEAvr is an average voltage of the working electrode voltage, n is a number of the working electrode voltages collected in the preset sampling period, i=1, 2, . . . , n, and represents a current i^{th} voltage, and WEVolᵢ represents an i^{th} working electrode voltage collected in the sampling period.

A standard deviation of the working electrode voltage is:

$$WEStd = \sqrt{\frac{1}{n} {\sum }_{i = 1}^{n} {( {WEVol}_{i} - WEAvr )}^{2}} ,$$

where WEStd is the standard deviation of the working electrode voltage.

A range value of the working electrode voltage is: WEMax_{diff}=Max(WEVolᵢ)−Min(WEVolᵢ), where WEMax_{diff} is the range value of the working electrode voltage, and Max(WEVolᵢ) is a maximum value of the working electrode voltage collected in the preset sampling period, and Min(WEVolᵢ) is a minimum value of the working electrode voltage collected in the preset sampling period.

An average voltage of the reference electrode voltage is:

$$REAvr = \frac{1}{n} {\sum }_{i = 1}^{n} {REVol}_{i} ,$$

where REAvr is the average voltage of the reference electrode voltage, i=1, 2, . . . , n, and represents a current i^{th} voltage, and REVolᵢ represents an i^{th} reference electrode voltage collected in the sampling period.

A standard deviation of the reference electrode voltage is:

$$REStd = \sqrt{\frac{1}{n} {\sum }_{i = 1}^{n} {( {REVol}_{i} - REAvr )}^{2}} ,$$

where REStd is the standard deviation of the reference electrode voltage.

A range value of the reference electrode voltage is: REMax_{diff}=Max(REVolᵢ)−Min(REVolᵢ), where REMax_{diff} is the range value of the working electrode voltage, Max(REVolᵢ) is a maximum value of the reference electrode voltage collected in a preset sampling period, and Min(REVolᵢ) is a minimum value of the reference electrode voltage collected in the preset sampling period.

Whether the average voltage, the standard deviation, and the range value are all within corresponding preset thresholds may be determined based on the following conditions:
Condition 1: WEAvr∈[WESetMinVoltage, WESetMaxVoltage], that is, whether the average voltage of the working electrode falls within a threshold of an acceptable range of the working electrode;
Condition 2: WEStd∈[WESetMinStd, WESetMaxStd], that is, whether the standard deviation of the working electrode voltage falls within a threshold of an acceptable range of the standard deviation of the working electrode voltage, and how to determine the acceptable range of the standard deviation of the working electrode voltage;
Condition 3: WEMax_{diff}<WESetMax_{diff}, that is, whether a maximum difference in the measurement of the working electrode voltage is less than a set upper limit value of the working electrode voltage;
Condition 4: REAvr∈[RESetMinVoltage, RESetMaxVoltage], that is, whether the average voltage of the reference electrode is within the threshold of the acceptable range of the average voltage of the reference electrode;
Condition 5: REStd∈[RESetMinStd, RESetMaxStd], that is, whether the standard deviation of the reference electrode voltage is within a threshold of an acceptable range of the standard deviation of the reference electrode voltage;
Condition 6: REMax_{diff}<RESetMax_{diff}, that is, whether the maximum difference in measurement of the reference electrode voltage is within an acceptable upper limit of the maximum difference of the reference electrode voltage; and

$$WorkVolSet = WEAvr - REAvr , \qquad Condition 7$$

WorkVolSet∈[WorkVolSetMinVoltage, WorkVolSetMaxVoltage], that is, the working voltage is set within an acceptable range of a working voltage setting, and the working voltage setting is usually calculated by using a difference between an average voltage of the working electrode (WEAvr) and an average voltage of the reference electrode (REAvr).

If it is determined that the average voltage, the standard deviation, and the range value all fall within the preset thresholds corresponding to the foregoing Condition 1 to Condition 7, it indicates that the physiological parameter detection apparatus 116 has a reliable measurement capability. In this case, the real-time working voltage and the real-time working current of the physiological parameter detection apparatus 116 can be obtained.

At step 310, a hardware-state vector is generated based on the at least one working attribute and a corresponding preset threshold.

To this end, for voltage-based evaluation, the working electrode voltage may be compared with a first preset voltage threshold, and the reference electrode voltage may be compared with a second preset voltage threshold to obtain a first voltage determination result. Additionally, a second voltage determination result may be determined based on voltage differences between the working electrode voltage and the reference electrode voltage, and between the working electrode voltage and the counter electrode voltage.

When either voltage difference exceeds a corresponding preset voltage difference threshold, the second voltage determination result is determined to indicate an abnormality.

Further, for current-based evaluation, the real-time working current may be compared with a second preset threshold to determine a current determination result. The current determination result may include multiple sub-results, including a first current determination result obtained by comparing the real-time working current with a threshold interval derived from detection sensitivity and physiological parameter range, a second current determination result obtained through signal filtering processing, and a third current determination result obtained through low-pass filtering and baseline drift analysis.

When any of the current determination sub-results indicates an abnormality, the current determination result is determined to indicate an abnormality.

Thus, the hardware-state vector is generated based on the voltage determination result and the current determination result and reflects whether the physiological parameter detection apparatus is operating within acceptable hardware conditions.

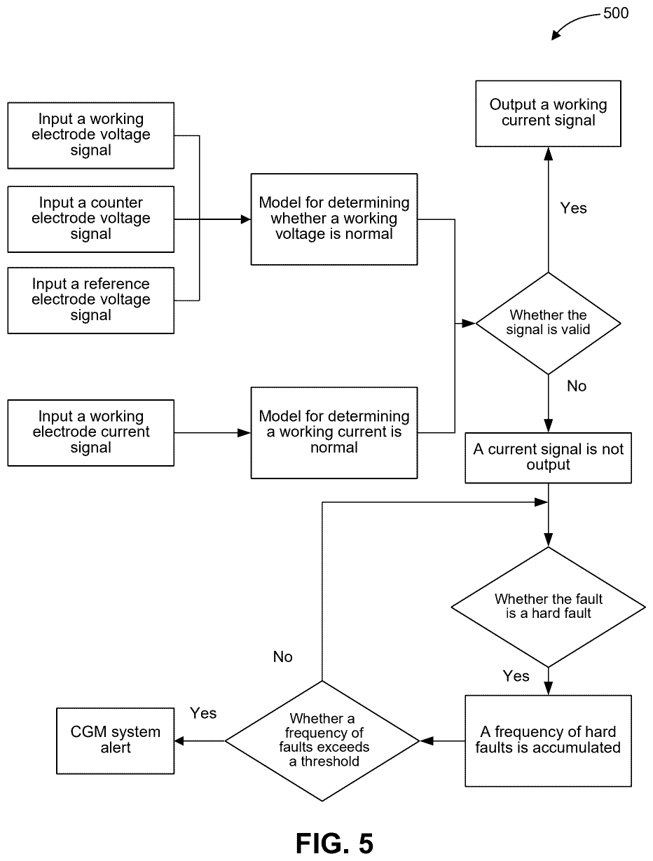

The physiological parameter detection apparatus 116 may be prone to be affected by problems such as noise, sensor performance drift, breakage of a screen-printed electrode, wearing compression, and bleeding short circuit when continuously detecting a physiological parameter, causing an anomaly of the physiological parameter detection apparatus 116. Therefore, to facilitate determining whether an abnormality occurs on the physiological parameter detection apparatus 116, in this application, as shown in FIG. 5, a real-time working voltage is compared with a first preset threshold to obtain a voltage determination result, and a real-time working current is compared with a second preset threshold to obtain a current determination result. By performing dual monitoring on both a voltage and a current, a working state of the physiological parameter detection apparatus 116 can be understood more comprehensively. In addition, for a wearable physiological parameter detection apparatus 116, abnormalities of the voltage and the current are found in time, so that an invalid signal collected by a faulty apparatus can be prevented from being continuously used, thereby helping to improve reliability of the physiological parameter detection apparatus 116.

Specifically, in an embodiment, based on a working circuit 400 shown in FIG. 4, the real-time working voltage includes a working electrode voltage WEVolᵢ, a reference electrode voltage REVolᵢ, and a counter electrode voltage CEVolᵢ. The first preset threshold includes a first preset voltage threshold, a second preset voltage threshold, and a preset dropout threshold. In this case, the comparing the real-time working voltage with the first preset threshold to determine a voltage determination result includes the following process:

The tested real-time working voltage is input to the following model for determining whether a working voltage is normal:

$${VolLoopResult}_{i} = func\_ProcessVol ( {WEVol}_{i} , {REVol}_{i} , {CEVol}_{i} )$$

where VolLoopResultᵢ is a voltage determination result.

According to the foregoing model, first, the working electrode voltage is compared with the first preset voltage threshold, and the reference electrode voltage is compared with the second preset voltage threshold, to determine a first voltage determination result. That is, in a voltage determining process, whether a voltage signal is stable is first determined by using a determination model. That is, whether the voltage values input by the working electrode and the reference electrode are stable values needs to be determined. When fluctuations generated by the input voltage signal WEVolᵢ and REVolᵢ exceed set thresholds, it indicates that the first voltage determination result is an abnormality. In addition, because there is a stable voltage difference in an electrochemical reaction process of the physiological parameter detection apparatus 116, in a process of detecting an abnormality, voltage differences between the working electrode voltage and the counter electrode voltage and between the reference electrode voltage and the counter electrode voltage further need to be determined, to determine a second voltage determination result according to the voltage difference and a corresponding preset voltage difference threshold. If there is an abnormal result in the first voltage determination result or the second voltage determination result, it indicates that the current voltage determination result VolLoopResultᵢ indicates an abnormality, error processing needs to be performed.

The working electrode voltage, the reference electrode voltage, and the counter electrode voltage are simultaneously monitored, and are compared with the preset voltage threshold. This process can comprehensively detect a voltage abnormal case in an electrochemical system. Such comprehensive detection helps to find and process a potential problem in time, thereby improving stability and reliability of the physiological parameter detection apparatus 116. Stability of the voltage signal, that is, whether the voltage value fluctuates within a stable range, is determined. This process can ensure that a voltage in the electrochemical system is in a stable state. A voltage difference affects a rate and a direction of the electrochemical reaction in the physiological parameter detection apparatus 116. Therefore, whether a current electrochemical reaction of the physiological parameter detection apparatus 116 operates normally can be determined in time by detecting a voltage difference between the working electrode voltage and the counter electrode voltage and between the reference electrode voltage and the counter electrode voltage and comparing the voltage difference with a preset voltage difference threshold, that is, finding out a voltage abnormality in time, thereby helping to reduce overall maintenance costs of the physiological parameter detection apparatus 116.

In an embodiment, referring to FIG. 4, the preset voltage difference threshold includes a first preset voltage difference threshold and a second preset voltage difference threshold, and the determining a second voltage determination result includes the following process:
a working electrode end (WE) and a counter electrode end (CE) are a circuit through which an electrochemical reaction generates a current. According to a principle of a virtual short circuit of an operational amplifier, a working electrode voltage of the working electrode end is equal to a voltage set by a Digital-to-Analog Converter (DAC). There is a stable voltage difference Vol_WR between the working electrode voltage and the reference electrode voltage, and the voltage of the reference electrode end (RE) is equal to the voltage set by the DAC according to the principle of the virtual short circuit of the operational amplifier, so Vol_WR is a stable value. Therefore, whether the first voltage difference between the working electrode voltage and the counter electrode voltage exceeds the first preset voltage difference threshold is determined by using a model for determining whether a working voltage is normal. That is, the first voltage difference is compared with the first preset voltage difference threshold to obtain a first comparison result. In addition, because the counter electrode end is connected to an output end of the operational amplifier, and a voltage is not fixed, but the voltage of the counter electrode end needs to be lower than the voltage of the reference electrode end and the voltage of the working electrode end, a second voltage difference between the reference electrode voltage and the counter electrode voltage needs to be within the second preset voltage difference threshold. Therefore, the second voltage difference needs to be compared with the second preset voltage difference threshold to obtain the second comparison result. A second voltage determination result VolLoopResultᵢ is determined as an abnormality if there is a result that exceeds a corresponding preset voltage difference threshold between the first comparison result and the second comparison result.

In the process, once the voltage difference exceeds the preset voltage difference threshold, an alerting signal can be sent immediately in this process, and the second voltage determination result is determined as an abnormality. Such a rapid early anomaly alert and response mechanism not only improves anomaly detection efficiency, but also helps to avoid a problem that incorrect physiological parameter detection data is used due to output of an invalid signal, thereby improving reliability of the physiological parameter detection apparatus 116.

In an embodiment, referring to FIG. 5, a flowchart of a process 500 of comparing a real-time working current with a second preset threshold to determine a current determination result is illustrated.

The tested real-time working current is input to the following model for determining whether a working current is normal:

$${CurrLoopResult}_{i} = func\_ProcessCurr ( {InCurr}_{i} )$$

where CurrLoopResultᵢ is a current determination result, and InCurrᵢ is a real-time working current.

Because the physiological parameter detection apparatus 116 is affected by circuit noise, contact, drift, compression, and the like, a current value corresponding to the real-time working current sometimes is not a current value that can reflect a physiological parameter detection value. Therefore, in this embodiment, to determine whether the real-time working current is affected by an interference factor to cause an abnormal case, a first scope value corresponding to detection sensitivity of the physiological parameter detection apparatus 116 is obtained, and a second scope value corresponding to the to-be-detected physiological parameter is obtained. Then, a threshold interval of the real-time working current is determined according to the first scope value and the second scope value. The determination model determines the real-time working current based on the threshold interval, to obtain a first current determination result.

For example, in a physiological parameter detection apparatus 116 such as a CGM, in a normal case, a working current of a sensor of the CGM and a glucose concentration of a human body have a linear directly proportional mapping relationship, and a conversion coefficient between the working current and the glucose concentration is defined as in-vivo sensitivity. This is specifically expressed as:

$$GLU = \frac{{I}_{w}}{{K}_{s}} = \frac{{InCurr}_{i}}{{K}_{s}} ,$$

where GLU is the glucose concentration of the human body, I_{w} is the working current of the CGM sensor, and Kₛ is the in-vivo sensitivity. It can be learned based on the expression that the real-time working current is InCurrᵢ=GLU*Kₛ. Because Ks is a sensitivity value of a glucose sensor, in a standardized production condition, there is a first scope value within a normal range, and in a human body system, GLU has a second scope value within a normal scope value. Therefore, it may be determined that a normal real-time working current InCurrᵢ has a determined scope value [MinInCurr, MaxIncurr] according to the first scope value and the second scope value. The determination model determines whether the input real-time working current InCurrᵢ exceeds a set threshold interval, so that the first current determination result may be obtained.

A fluctuation of a blood glucose value of the human body is a low-frequency signal. Therefore, signal filtering processing is performed on the real-time working current, and a second current determination result is determined according to a processed real-time working current and a preset current threshold, so that a case in which the real-time working current exceeds the threshold caused by cases such as high-frequency vibration and sudden rising and sudden falling can be determined. In addition, for cases such as performance attenuation or damage to an outer membrane that occur on the sensor, signal drift occurs in the real-time working current in a relatively long time. Therefore, low-pass filtering needs to be performed on the real-time working current, to obtain a baseline of the real-time working current. A third current determination result may be determined according to drift data of the baseline and a corresponding preset drift data threshold. It may be understood that when the baseline drifts beyond a normal fluctuation within a long-time range, the third current determination result indicates an abnormality, and error processing needs to be performed. If there is an abnormal result in the foregoing first current determination result, second current determination result, or third current determination result, a current determination result CurrLoopResultᵢ of a real-time working current indicates an abnormality, that is, an output of a model for determining whether a working current is normal is an abnormality.

Further, in an embodiment, the method further includes: it is determined that the real-time working current is an invalid signal if the voltage determination result or the current determination result is an abnormality. As shown in FIG. 5, when the voltage determination result output by a model for determining whether a working system is normal is an abnormality or the current determination result output by the model for determining whether a working current is normal is an abnormality, a real-time working current signal is an invalid signal. When an abnormal case occurs, the real-time working current that can reflect the physiological parameter detection data is determined as an invalid signal, so that application of an abnormal detection result can be avoided, thereby ensuring reliability of the physiological parameter detection apparatus 116, and avoiding further use of an erroneous result.

More specifically, as shown further in FIG. 5, when a signal is determined to be invalid, the current signal is not output. It is further determined whether the fault that leads to the invalid signal is a fault associated with hardware failure, which may be referred to as a hard fault or a hard anomaly. When a hard fault or a hard anomaly is determined to occur, a frequency of hard faults is accumulated. The frequency of hard faults may be further compared to a threshold. If the frequency of hard faults does exceed the threshold, a CGM system alert may be triggered.

At step 312, it is determined whether an anomaly exists in the output of the physiological parameter detection apparatus by jointly evaluating the signal-state vector and the hardware-state vector corresponding to the current moment. Step 312, thus, provides for a decision-fusion stage in which results of signal-level analysis and apparatus-level analysis are correlated to produce a final determination regarding validity or abnormality of the human vital sign signal. The first state vector and the second state vector may be indicative of an anomaly in a human vital sign signal at the current moment.

As shown in FIG. 2, if no abnormal signal exists, the human vital sign signal is directly outputted, and if an abnormal signal exists, whether the abnormal signal is repairable is further determined. The abnormal signal may be determined based on whether the estimated signal vector falls within an estimated signal interval. If the estimated signal vector does not fall within the estimated signal interval, the abnormal signal exists.

Specifically, for a human vital sign signal at a single moment, if a first state vector corresponding to the human vital sign signal represents that a change state during preprocessing does not change (that is, the first state vector is recorded as 1 in the set corresponding to the first state vector), and a second state vector corresponding to the human vital sign signal represents that the second state vector falls within the estimated signal interval (that is, the second state vector is recorded as 1 in the set corresponding to the second state vector), the human vital sign signal at the single moment is used as a valid signal, and a valid sequence is recorded as CgmValidₙ=CgmStatusₙ∩CgmInGapₙ.

$${CgmValid}_{i} = \{ \begin{matrix}1 , {CgmInGap}_{i} = 1 and {CgmStatus}_{i} = 1 \\ 0 , otherwise\end{matrix} ,$$

where CgmStatusᵢ represents the first state vector, and CgmInGapᵢ represents the second state vector.

Based on this, a vector XorCgmVaild=CgmValid⊕M is set, where a vector M=[1, 1, 1 . . . ], and a vector CgmValid is a vector composed of a valid signal CgmValidᵢ.

If a count of continuous 1 in the vector XorCgmVaild is not less than a preset threshold Err_Thd, it indicates that the signal is continuously preprocessed and updated or does not fall within the estimated signal interval, unrepairable anomaly information is outputted. Otherwise, it is determined that the anomaly is repairable, and no anomaly information is outputted. A code repairing process may be similar to the signal preprocessing process described above, that is, an excessively large or small signal value is repaired to be within a proper interval.

The signal-state vector reflects whether the estimated vital sign signal is consistent with expected physiological behavior, as determined through filtering-based estimation and constraint-model-based interval validation. The hardware-state vector reflects whether one or more working attributes of the physiological parameter detection apparatus, such as real-time working voltage or real-time working current, are operating within corresponding preset thresholds. By combining these two vectors, the system avoids reliance on signal behavior or hardware condition in isolation, thereby improving robustness of anomaly detection under real-world operating conditions.

In some embodiments, each of the signal-state vector and the hardware-state vector may include a discrete state value for the current moment, such that the state value may be represented by a first preset value (e.g., indicating abnormality) or a second preset value (e.g., indicating normality). The step 312, for example, may be performed using logical evaluation rules applied to these state values.

At step 314A, the system 100 may determine that an anomaly exists in the human vital sign signal at the current moment when either of the following conditions is satisfied:
the state value of the signal-state vector corresponding to the estimated vital sign signal at the current moment is the first preset value, or
the state value of the hardware-state vector corresponding to the current moment is the first preset value.

To this end, a signal-state vector state value of the first preset value may indicate that the estimated vital sign signal does not satisfy one or more signal-level validity conditions, such as falling outside a predicted signal interval derived from recent signal statistics. Accordingly, the condition suggests that the signal behavior is inconsistent with expected physiological trends and may represent noise corruption, signal dropout, saturation, or other abnormal signal phenomena.

Similarly, a hardware-state vector state value of the first preset value indicates that at least one working attribute of the physiological parameter detection apparatus exhibits abnormal behavior relative to a corresponding preset threshold. For example, an abnormal voltage determination result, an abnormal current determination result, or a persistent baseline drift may cause the hardware-state vector to indicate abnormality. In such cases, even when the signal behavior appears superficially plausible, the underlying apparatus conditions may compromise signal reliability.

Once an anomaly is determined at step 314A, the system 100 may proceed to subsequent processing operation, including anomaly type classification (e.g., soft anomaly versus hard anomaly), persistence evaluation across multiple moments, signal repair or suppression, and/or alarm generation.

Additionally, at step 314A, a anomaly type of the physiological parameter detection apparatus 116 may be determined according to the voltage determination result and the current determination result.

Anomalies existing in the current physiological parameter detection apparatuses 116 may be classified into two types: hard anomalies and soft anomalies. The hard anomalies are mainly because a problem occurs in the sensor or the physiological parameter detection apparatus 116, and in this case, the working current signal is an invalid signal. The soft anomalies are mainly generated in a use process of the physiological parameter detection apparatus 116, and are mostly correctable anomalies. As shown in FIG. 5, whether the real-time working current is valid can be determined after the voltage determination result and the current determination result are obtained. In this case, a specific type of an anomaly can be further determined by combining the voltage determination result and the current determination result, thereby facilitating targeted maintenance for the anomaly corresponding to the physiological parameter detection apparatus 116.

Specifically, in an embodiment, the anomaly type includes a soft anomaly and a hard anomaly, and the determining an anomaly type of the physiological parameter detection apparatus 116 according to the voltage determination result and the current determination result includes the following process: if the voltage determination result is an abnormality, that is, the voltage determination result VolLoopResultᵢ is an abnormality, it may be determined that the anomaly type is the hard anomaly. It may be learned based on the foregoing content that the voltage determination result VolLoopResultᵢ is a result obtained after the real-time working voltage is processed by a function func_ProcessVol in the foregoing process. If the third current determination result is an abnormality, that is, a drift case occurs, the anomaly type may alternatively be determined as the hard anomaly. It may be learned based on the foregoing content that the current determination result CurrLoopResultᵢ is a result obtained after the real-time working current is processed by a function func_ProcessCurr in the foregoing process. If the first current determination result or the second current determination result indicates an abnormality, whether the abnormality is corrected within a preset time step needs to be detected. If the abnormality can be corrected within the preset time step, the anomaly type is determined as the soft anomaly. Otherwise, if the abnormality is not corrected within the preset time step, the anomaly type is determined as the hard anomaly.

By combining the voltage determination result and the current determination result, whether the soft anomaly or the hard anomaly occurs in the physiological parameter detection apparatus 116 can be accurately distinguished. However, by detecting whether the abnormality is corrected within the preset time step, the soft anomaly can be dynamically monitored and determined. This method not only considers instantaneous interference, but also considers a possibility of a persistent anomaly, thereby improving accuracy and flexibility of anomaly determination. By means of the foregoing accurate anomaly type determination and a timely anomaly processing, problems of instability and reduced reliability of the physiological parameter detection apparatus 116 caused by an anomaly can be effectively reduced, thereby helping to ensure continuous and accurate operation of the physiological parameter detection apparatus 116.

Further, in an embodiment, as shown in FIG. 5, after determining the anomaly type of the physiological parameter detection apparatus 116, the method further includes: counting a number of times the physiological parameter detection apparatus 116 has a hard anomaly, and outputting an alerting signal to the physiological parameter detection apparatus 116 if the counted number of times exceeds a preset number threshold. A number of times of hard anomalies is counted, and when the number of times of anomalies reaches or exceeds a threshold, an alerting signal is output to remind maintenance personnel to perform checking and maintenance in time, thereby avoiding anomalies of a device at a critical time, and ensuring continuous and stable operation of the device. In addition, frequent hard anomalys may alternatively mean that the device has a serious quality problem or is about to reach a service life. Therefore, outputting the alerting signal in time may prompt relevant personnel to take measures, for example, replace the device or perform more in-depth technical maintenance, thereby reducing risks and losses caused by the anomaly of the physiological parameter detection apparatus 116.

At step 314B, the system 100 may determine that the human vital sign signal at the current moment is a valid code when both of the following conditions are satisfied:
the state value of the signal-state vector corresponding to the estimated vital sign signal at the current moment is the second preset value, and
the state value of the hardware-state vector corresponding to the current moment is the second preset value.

In such cases, the signal-state vector indicating the second preset value signifies that the estimated vital sign signal falls within the predicted signal interval and is consistent with recent physiological trends captured within the preset time window. The condition, thus, indicates that the signal behavior aligns with expectations derived from filtering-based estimation and constraint-model analysis.

Simultaneously, the hardware-state vector indicating the second preset value signifies that the working attributes (i.e., working voltage and working current) of the physiological parameter detection apparatus 116 are operating within their respective preset thresholds. The condition, thus, further indicates that the apparatus 116 is functioning normally from a hardware and electrical standpoint.

When both vectors concurrently indicate the second preset value, the system 100 may determine that the human vital sign signal at the current moment is reliable and suitable for downstream use (e.g., display, storage, clinical decision support, or further analytics). The signal may be recorded as a valid code and, in some embodiments, may be used to update the optimal estimation result used by the filtering model for subsequent moments.

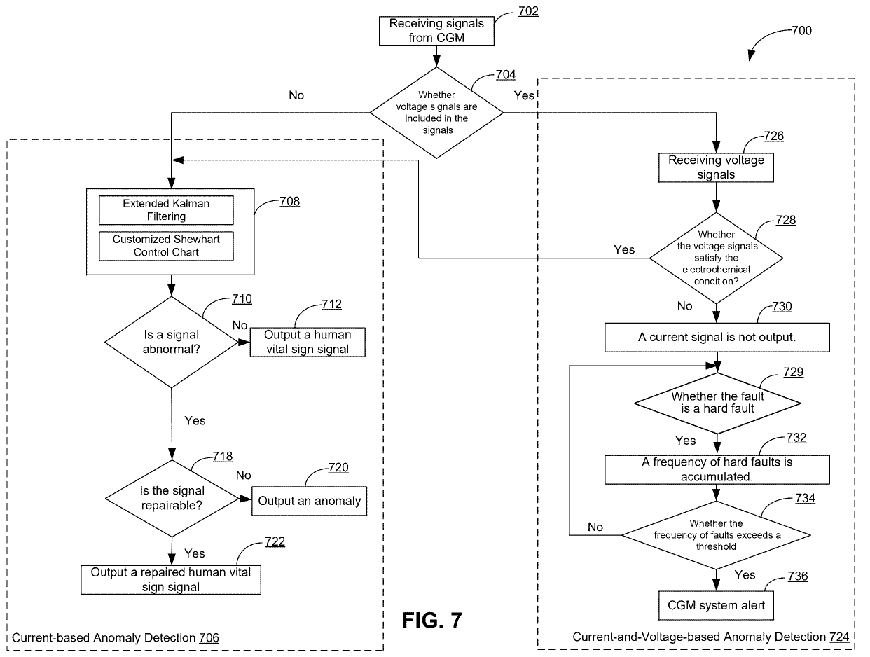

FIG. 7 shows another schematic diagram illustrating a flow chart 700 that integrates the logical framework of anomaly type detection and the method for detecting an anomaly in a human vital sign signal. Each block may indicate an executable operation.

At block 702, one or more signals are received from the physiological parameter detection apparatus 116 (e.g., the CGM). The signals may include current signals and voltage signals. Sometimes, the voltage signals may be missing. Thus, at decision block 704, it is determined whether voltage signals respectively from the working electrode (WE), the counter electrode (CE), and the reference electrode (RE) are included in the signals from the physiological parameter detection apparatus 116. If the decision is YES (i.e., the voltage signals are included), a current-and-voltage-based anomaly detection 724 may be implemented. If the decision is NO (i.e., the voltage signals are missing), a current-based anomaly detection 706 may be implemented.

Based on a determination that the voltage signals are missing, the signals may be input to a filtering module 708. As described above, the extended Kalman filtering and the customized Shewhart control chart may be implemented to process the signals. The processed signals may be input to decision block 710 to check if any of the processed signals is abnormal. If the processed signals are normal, a human vital sign signal may be output at block 712.

If any of the processed signals is determined to be abnormal, the abnormal signal may be determined to be repairable or not at decision block 718. If the abnormal is determined to be not repairable, an anomaly may be output at block 720.

If the abnormal signal is determined to be repairable, the abnormal signal may be further repaired and a repaired human vital sign signal may be output at block 722.

On the other hand, based on a determination that the voltage signals are included in the signals from the physiological parameter detection apparatus 116, the voltage signals, along with other signals, may be examined in the current-and-voltage-based anomaly detection 724 at block 726. First, at decision block 728, it is determined whether the voltage signals satisfy a electrochemical reaction condition (alternatively referred to as “whether the signal is valid”). For example, as mentioned above, the working electrode voltage, the reference electrode voltage, and the counter electrode voltage are simultaneously monitored, and are compared with the preset voltage threshold, which may comprehensively detect a voltage abnormal case in an electrochemical system.

Based on a determination that the voltage signals satisfy the electrochemical reaction condition, the signals including the voltage signals may be fed back to the current-based anomaly detection 706 and go through the operations as described above.

If the voltage signals are determined to fail the electrochemical reaction condition, at block 730, current signals are not output and a fault type may be determined (e.g., hardware fault or software fault) at decision block 729. If it is determined to be a hardware fault, a frequency of hard faults may be accumulated at block 732.

At decision block 734, it is determined whether the frequency of faults exceeds a threshold. If the decision is Yes, a CGM system alert may be triggered. If the decision is No, the process may restart at block 730 or block 729.

An embodiment of this application provides an anomaly detection system for a physiological parameter detection apparatus. The system includes:
one or more sensors,
at least one processor; and
a memory in communication connection with the at least one processor, where
the memory stores an instruction that can be executed by the at least one processor, and the instruction is executed by the at least one processor to enable the at least one processor to perform the method of detecting an anomaly of a physiological parameter detection apparatus, according to any one of the foregoing items.

An embodiment of this application provides a non-volatile storage medium, storing a computer executable instruction, where the computer executable instruction is set to be capable of performing method of detecting an anomaly of a physiological parameter detection apparatus, according to any one of the foregoing items.

The embodiments of this application are all described in a progressive manner, for same or similar parts in the embodiments, refer to such embodiments, and descriptions of each embodiment focus on a difference from other embodiments. In particular, for the embodiments of the device and the non-volatile computer storage medium, the embodiments are basically similar to the method embodiments and therefore are only briefly described, and for the associated part, refer to the method embodiments.

Specific embodiments of this application are described above. Other embodiments fall within the scope of the appended claims. In some embodiments, the actions or steps recorded in the claims may be performed in sequences different from those in the embodiments and an expected result may still be achieved. In addition, the processes depicted in the accompanying drawings is not necessarily performed in the specific order or successively to achieve an expected result. In some implementations, multitasking and parallel processing may be feasible or beneficial.

The foregoing descriptions are merely one or more embodiments of this application, but are not intended to limit this application. For a person skilled in the art, various modifications and changes may be made to one or more embodiments of this application. Any modification, equivalent replacement, or improvement made within the spirit and principle of the one or more embodiments of this application fall within the scope of the claims of this application.

What is claimed is:

1. A method of detecting an anomaly of a physiological parameter detection apparatus, the method comprising:
receiving one or more mixed signals from the physiological parameter detection apparatus;
determining whether the mixed signals include at least one real-time working voltage;
based on a determination that the mixed signals include the at least one real-time working voltage, implementing a current-and-voltage-based anomaly detection process; and
based on a determination that the mixed signals do not include the at least one real-time working voltage, implementing a current-based anomaly detection process.

2. The method of claim 1, wherein
the current-based anomaly detection process includes:
receiving, from the physiological parameter detection apparatus, a human vital sign signal indicative of a physiological parameter;
determining, using a filtering model, an estimated vital sign signal at a current moment based on the human vital sign signal at a previous moment;
generating a signal-state vector based on at least one of the human vital sign signal and the estimated vital sign signal;
the current-and-voltage-based anomaly detection process include:
acquiring at least one working attribute of the physiological parameter detection apparatus;
generating a hardware-state vector based on the at least one working attribute and a corresponding preset threshold; and
determining whether an anomaly exists in the output of the physiological parameter detection apparatus based on the signal-state vector and the hardware-state vector.

3. The method of claim 2, further comprising:
determining, through a constraint model, a predicted signal interval at the current moment based on a mean and a standard deviation of the human vital sign signal within a preset time window,
wherein generating the signal-state vector comprises:
determining whether the estimated vital sign signal falls within the predicted signal interval; and
wherein determining whether the anomaly exists comprises:
evaluating a state value of the signal-state vector corresponding to the current moment, and determining that an anomaly exists when the state value is equal to the second state value.

4. The method of claim 2, wherein the at least one working attribute comprises the real-time working voltage and a real-time working current of the physiological parameter detection apparatus.

5. The method of claim 4,
wherein the real-time working voltage comprises a working electrode voltage, a reference electrode voltage, and a counter electrode voltage, and
wherein the preset threshold corresponding to the real-time working voltage comprises a first preset voltage threshold and a second preset voltage threshold,
wherein the method further comprises:
comparing the working electrode voltage with the first preset voltage threshold, and comparing the reference electrode voltage with the second preset voltage threshold, to determine a first voltage determination result;
determining a second voltage determination result based on the working electrode voltage, the reference electrode voltage, and the counter electrode voltage; and
determining the anomaly in the output of the physiological parameter detection apparatus, when there is an abnormal result in the first voltage determination result or the second voltage determination result.

6. The method of claim 5, wherein a first preset threshold corresponding to the real-time working voltage comprises a first preset voltage difference threshold and a second preset voltage difference threshold, wherein determining the second voltage determination result comprises:
determining a first voltage difference between the working electrode voltage and the reference electrode voltage,
determining a second voltage difference between the working electrode voltage and the counter electrode voltage; and
determining that the second voltage determination result indicates an abnormality when the first voltage difference is greater than the first preset voltage difference threshold or the second voltage difference is greater than the second preset voltage difference threshold.

7. The method of claim 4, further comprising:
comparing the real-time working current with a second preset threshold to determine a current determination result,
the method further comprising:
obtaining a first scope value corresponding to detection sensitivity of the physiological parameter detection apparatus, and obtaining a second scope value corresponding to a to-be-detected physiological parameter;
determining a threshold interval of the real-time working current according to the first scope value and the second scope value, and determining the real-time working current based on the threshold interval, to obtain a first current determination result;
performing signal filtering processing on the real-time working current, and determining a second current determination result according to the processed real-time working current and a preset current threshold;
performing low-pass filtering processing on the real-time working current to obtain a baseline of the real-time working current, and determining a third current determination result based on drift data of the baseline and a corresponding preset drift data threshold; and
determining that the current determination result indicates an abnormality if there is an abnormal result in the first current determination result, the second current determination result, or the third current determination result.

8. The method of claim 7, further comprising:
determining an anomaly type for the anomaly, the wherein the anomaly type comprises a soft anomaly and a hard anomaly, wherein determining the anomaly type comprises:
determining that the anomaly type is the hard anomaly if at least one of the first voltage determination result or the second voltage determination result is an abnormality;
determining that the anomaly type is the hard anomaly if the third current determination result is an abnormality;
detecting whether the abnormality is corrected within a preset time step if the first current determination result or the second current determination result is an abnormality;
determining that the anomaly type is the soft anomaly if the abnormality is corrected within the preset time step; and
determining that the anomaly type is the hard anomaly if the abnormality is not corrected within the preset time step.

9. The method of claim 8, wherein determining the anomaly type further comprises:
counting a number of times the physiological parameter detection apparatus has a hard anomaly, and outputting, by the physiological parameter detection apparatus, an alarm signal if the counted number of times exceeds a preset number threshold.

10. The method of claim 4, wherein prior to the obtaining the real-time working voltage and a real-time working current of the physiological parameter detection apparatus, the method further comprises:
collecting an initial voltage of the physiological parameter detection apparatus according to a preset sampling period;
calculating an average voltage, a standard deviation, and a range value that correspond to the initial voltage, and determining whether the average voltage, the standard deviation, and the range value are all within corresponding preset thresholds; and
if the average voltage, the standard deviation, and the range value are all within the corresponding preset thresholds, obtaining the real-time working voltage and the real-time working current of the physiological parameter detection apparatus.

11. The method of claim 4, further comprising:
determining that the real-time working current is an invalid signal if the voltage determination result or the current determination result is an abnormality.

12. The method of claim 2, wherein determining the estimated vital sign signal comprises:
linearizing a state equation of the filtering model to obtain a linear state equation; and
obtaining the estimated vital sign signal at the current moment based on an optimal estimation result of the human vital sign signal at the previous moment by using the linear state equation.

13. The method of claim 2, wherein determining the predicted signal interval comprises:
calculating a signal standard deviation and a signal mean based on the human vital sign signal within the preset time window; and
inputting the signal standard deviation, the signal mean, the human vital sign signal at the previous moment, and the estimated vital sign signal to the constraint model, and determining, by the constraint model, the predicted signal interval at the current moment.

14. The method of claim 13, wherein the predicted signal interval at the current moment comprises a maximum critical value and a minimum critical value;
a calculation formula of the maximum critical value is:
$${upLimit\_CgmSignal}_{i} = G ( {Pro\_CgmSignal}_{i - 1} , Ave\_Pro {\_CgmSignal}_{N} , {\overline{x}}_{k + 1 \vert k} ) + γ * Std\_Pro {\_CgmSignal}_{N}$$
a calculation formula of the minimum critical value is:
$${LowLimit\_CgmSignal}_{i} = G ( {Pro\_CgmSignal}_{i - 1} , Ave\_Pro {\_CgmSignal}_{N} , {\overline{x}}_{k + 1 \vert k} ) - β * Std\_Pro {\_CgmSignal}_{N} ,$$
wherein Pro_CgmSignalᵢ₋₁ represents the human vital sign signal at the previous moment; Ave_Pro_CgmSignal_{N} represents the signal mean; Std_Pro_CgmSignal_{N} represents the signal standard deviation; and γ and β represent preset coefficients.

15. The method of claim 2, wherein the preset signal value comprises a first signal value, a second signal value, and a third signal value,
wherein, before the generating the signal-state vector, the method further comprises:
preprocessing the human vital sign signal based on the first signal value, the second signal value, and the third signal value, to obtain a pre-processed vital sign update signal; and
correspondingly, the generating the signal-state vector based on a human vital sign signal and a preset signal value comprises:
generating the signal-state vector based on the vital sign update signal.

16. The method according to claim 15, wherein preprocessing the human vital sign signal comprises:
updating the human vital sign signal to the first signal value if the human vital sign signal is greater than the first signal value;
updating the human vital sign signal to a sum value of the human vital sign signal at the previous moment and the second signal value if the human vital sign signal is greater than the human vital sign signal at the previous moment and a difference is greater than the second signal value; and
updating the human vital sign signal to a difference between the human vital sign signal at the previous moment and the third signal value if the human vital sign signal is less than the human vital sign signal at the previous moment and an absolute value of the difference is greater than the third signal value.

17. The method of claim 15, wherein generating the signal-state vector comprises:
determining a state value corresponding to the vital sign update signal as a first preset value if the human vital sign signal is greater than or equal to the first signal value or if the vital sign update signal is 0;
determining the state value corresponding to the vital sign update signal as the first preset value if the human vital sign signal is greater than the human vital sign signal at the previous moment and an absolute value of a difference is greater than or equal to the second signal value;
determining the state value corresponding to the vital sign update signal as the first preset value if the human vital sign signal is less than the human vital sign signal at the previous moment and the absolute value of the difference is greater than or equal to the third signal value;
determining the state value corresponding to the vital sign update signal as a second preset value if the human vital sign signal is not less than the human vital sign signal at the previous moment, or the absolute value of the difference is less than the third signal value; and
generating the signal-state vector based on the state value of each vital sign update signal.

18. The method of claim 2, wherein generating the signal-state vector comprises:
determining a state value corresponding to the estimated vital sign signal as a second preset value if the estimated vital sign signal falls within the predicted signal interval;
determining the state value corresponding to the estimated vital sign signal as a first preset value if the estimated vital sign signal does not fall within the predicted signal interval; and
generating the signal-state vector based on the state value of each estimated vital sign signal.

19. The method of claim 2, wherein determining the anomaly comprises:
if the state value of the signal-state vector corresponding to the estimated vital sign signal is a first preset value or a state value of the hardware-state vector is the first preset value, determining that an anomaly exists in the human vital sign signal at the current moment; and
if the state value of the signal-state vector corresponding to the estimated vital sign signal is a second preset value and the state value of the hardware-state vector is the second preset value, determining that the human vital sign signal at the current moment is a valid code.

20. The method of claim 19, further comprises:
determining an optimal estimation result corresponding to the human vital sign signal at the current moment, and acquiring a number of consecutive occurrences of determining that the human vital sign signal at the current moment is a valid code;
outputting the human vital sign signal at the current moment as an unrepairable signal if the number of consecutive occurrences is greater than or equal to a third preset value; and
outputting the human vital sign signal at the current moment as a repairable signal if the number of consecutive occurrences is less than the third preset value.

21. The method of claim 14, wherein generating the signal-state vector comprises:
determining a state value corresponding to the vital sign update signal as a first preset value if the vital sign update signal is different from the human vital sign signal, or if the human vital sign signal is zero;
determining the state value corresponding to the vital sign update signal as a second preset value if the vital sign update signal is equal to the human vital sign signal; and
generating the signal-state vector based on the state value corresponding to each vital sign update signal.
